(19) United States
(12) Patent Application Publication
(10) Pub. No.: US 2008/0200732 A1
(43) Pub. Date: Aug. 21, 2008

(54) Novel Intermediates Useful for the Preparation of Coenzymes, Process for the Preparation of Novel Intermediates and an Improved Process for the Preparation of Coenzymes

(75) Inventors: Abhay Upare, Mumbai (IN); Nitin Yeshwant Pawar, Mumbai (IN); Ganesh Wagh, Mumbai (IN); Amit Chavan, Mumbai (IN); Mita Roy, Mumbai (IN); Hariharan Sivaramakrishnan, Mumbai (IN)

Correspondence Address: HAMRE, SCHUMANN, MUELLER & LARSON, P.C., P.O. BOX 2902, MINNEAPOLIS, MN, 55402-0902, US

(73) Assignee: Nicholas Piramal India Limited, Mumbai (IN)

(21) Appl. No.: 11/994,797

(22) PCT Filed: Jun. 21, 2006
(86) PCT No.: PCT/IB06/52009

§ 371 Date: Mar. 4, 2008

(30) Foreign Application Priority Data
Jul. 6, 2005 (IN) 805/MUM/2005

(51) Int. Cl. C07C 45/61; C07C 43/215; C07C 41/30; C07C 43/225

(52) U.S. Cl. 568/322; 568/654; 568/649

(57) ABSTRACT

The present invention relates to novel intermediates for the preparation of coenzymes, processes for the preparation of the intermediates and an improved process for the preparation of Coenzymes. The present invention particularly relates to an improved process for the preparation of Coenzyme Q, more particularly for Conenzyme Q₉ and Coenzyme Q₁₀. Still more particularly this invention relates to regio and stereo controlled process for the preparation of Coenzyme Q₉ and Coenzyme Q₁₀ of the formula I

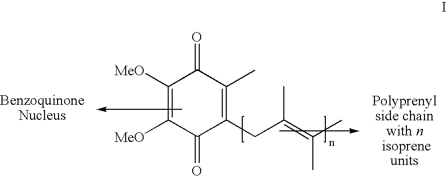

where n=9 (Coenzyme CoQ₉), and where n=10. (Coenzyme CoQ₁₀)

### FIELD OF INVENTION

The present invention relates to an improved process for the preparation of Coenzymes. The invention also relates to novel intermediates for the preparation of coenzymes, and process for the preparation of the intermediates. The present invention particularly relates to an improved process for the preparation of Coenzyme Q, and more particularly Conenzyme Q₉ and Coenzyme Q₁₀. Still more particularly this invention relates to regio and stereo controlled process for the preparation of Coenzyme Q₉ and Coenzyme Q₁₀ of formula I.

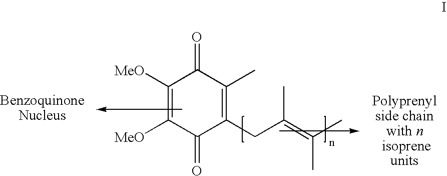

where n=9 (Coenzyme CoQ₉), and where n=10 (Coenzyme CoQ₁₀).

In the description given below the Coenzyme CoQ₉ is referred to as formula I₉ and Coenzyme CoQ₁₀ as formula I₁₀

### BACKGROUND AND PRIOR ART

These coenzymes belong to the class of ubiquinones that occur in all aerobic organisms from bacteria to plants and animals—the name ubiquinone suggests its ubiquitous occurrence. They are involved in mitochondrial processes such as respiration and act as antioxidants.

The present invention also provides novel Grignard reagent that is useful for the preparation of above mentioned coenzymes and a process for its preparation.

The coenzyme Q₁₀ in human has 10 isoprenoid units, and termed as CoQ₁₀. CoQ₁₀ is present in virtually every cell in the human body and is known as the “miracle nutrient”, and plays a vital role in maintaining human health and vigor, maintenance of heart muscle strength, enhancement of the immune system, quenching of free radical in the battle against aging to name a few (“The miracle nutrient coenzyme” Elsevier/North—Holland Biomedical Press, New York, 1986; “Coenzyme Q: Bioechemistry, Bioenergetics, and clinical Applications of Ubiquinone” Wiley, New York, 1985; “Coenzyme Q, Molecular Mechanism in Health and Disease” CRC press).

As depicted above Coenzyme Q₉ and Coenzyme Q₁₀ of the formula I have 2,3-dimethoxy-1,4-benzoquinone nucleus as a head group with a side chain of n isoprene units. The poly prenyl side chain in Coenzyme Q has all-trans configuration. One of the methods of synthesis of these Coenzymes is coupling of the “benzoquinone nucleus” with the “polyprenyl side chain” of solanesol of the formula 3a₉, where x=—OH and decaprenol of the formula 3a₁₀, where x=—OH. with retention of its original double bond geometry.

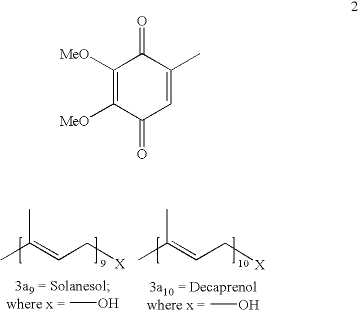

Various methods for introducing polyprenyl side chain into quinone nucleus, to prepare Coenzymes are found in literature. These methods involve functionalisation of the two coupling partners, the “quinone nucleus” and the “polyprenyl chain”.

Method 1: Polyprenyl alcohol and hydroquinone using zinc chloride as catalyst; reported in Huanxue Yu Nianhe (2002), 6 267(2002) which is shown in the Scheme 1 given below

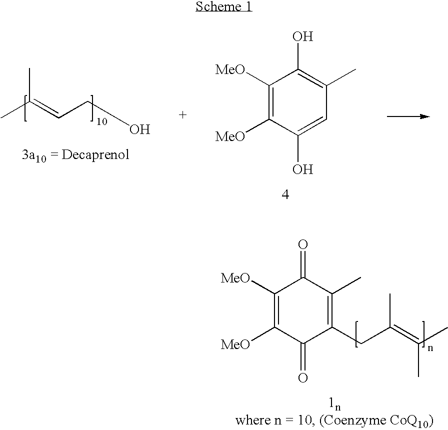

Decaprenol of the formula 3a₁₀ (1.8 g) dissolved in ether is treated with 2,3-dimethoxy-5-methyl benzohydroquinone of the formula 4, zinc chloride (anhydrous, 0.28 g), glacial acetic acid (0.02 ml) and stirred for 2 hours under nitrogen atmosphere. Ferric chloride solution is added to the above reaction mixture, stirred for ten minutes. The ethereal layer is then separated, dried and evaporated to give 2.2 g of crude CoQ₁₀ which is purified by column chromatography to give 0.56 g of the pure CoQ₁₀ of the formula I₁₀ with an overall yield of 20% (mp 45-46° C., Lit. mp 48-50° C.).

Low melting point obtained indicates the presence of cis-isomer and thereby making the process not stereoselective. The yield is also too low for commercialization of the process.

Method 2: By making π-Allyl Nickel bromide complex and protected quinone nucleus; reported in Bull. Chem. Soc. Jpn 47, 3098(1974), U.S. Pat. No. 3,896,153(1975) which is shown in scheme 2

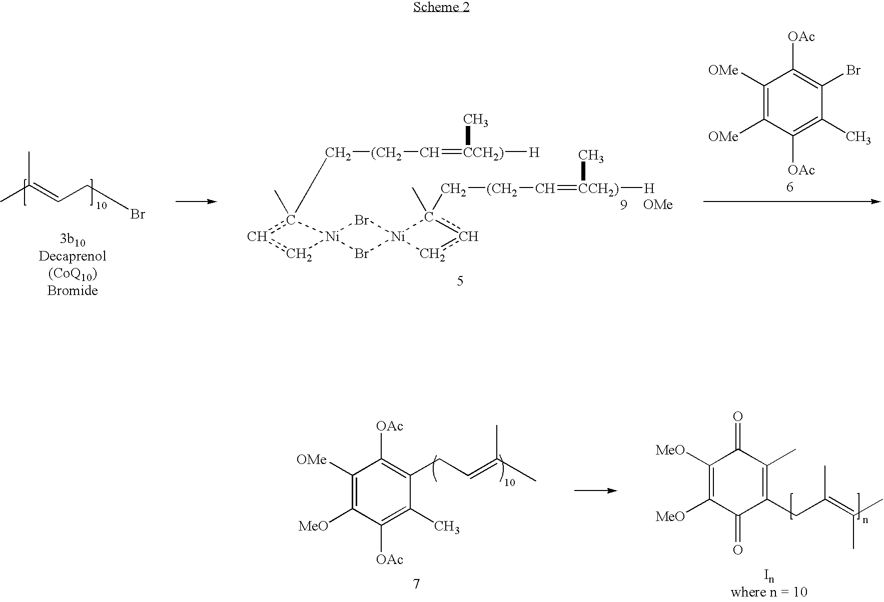

Nickel tetracarbonyl 4.5 g (15% solution in benzene) is treated with decaprenyl bromide of the formula 3b₁₀ 10.0 g (15% solution in Benzene) at 50° C. for 4-4.5 hrs. The solution is cooled to below 10° C. and the benzene and excess nickel carbonyl is removed under reduced pressure. Decaprenyl nickel bromide of the formula 5 thus formed is then reacted with 6-bromo-2,3-dimethoxy-5-methyl-1,4-hydroquinone diacetate of the formula 6 in 30 ml of hexamethyl phosphoramide at 75° C. for 7 hours yielding 2.2 g of condensed product of the formula 7 with 40% yield. The condensed product of the formula 7 (0.8 g) is added to a suspension of lithium aluminum hydride in 20 ml of dry ether and refluxed for 24 hours. The excess lithium aluminum hydride is decomposed and the product hydroquinone is extracted in ether.

The hydroquinone is oxidized with aqueous ferric chloride at room temperature for 3 hour to give the final product CoQ₁₀ which is further purified by column chromatography to yield the COQ₁₀ of the formula I₁₀ with mp 20-22° C. (Lit. mp 48-50° C.) with 69% yield.

Author attributes the low melting point to the presence of cis isomer. The process is therefore not stereoselective. Further, the nickel tetracarbonyl used in the process is highly flammable, has the risk of explosion and highly toxic chemical, and cannot be used industrially. The overall yield of the process is only 27.6%. The process is therefore not suitable for industry.

Method 3: From allyl-stannyl and unprotected quinone using borontrifluoride etherate; reported in J. Org. Chem. 45, 4077 (1980), Chemistry Letters 885(1979) as shown in scheme 3.

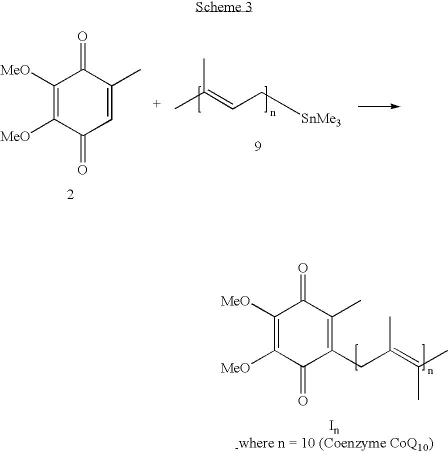

Trimethylstannyl lithium in tetrahydrofuran is slowly added to decaprenyl bromide of the formula 3b₁₀ at −78° C. to −60° C. and the reaction mixture is allowed to warm to room temperature. The reaction mixture is quenched in brine and the organic layer evaporated to form trimethyl decaprenyl stannanes of the formula 9. The stannyl reagent (0.42 mmol) in a mixture of methylene dichloride (25 ml) and isooctane (1 ml) is added to 2,3-dimethoxy-5-methylbenzoquinone (0.111 g, 0.61 mmol) and borontrifluoride etherate (2.6 mmol) in a mixture of methylene chloride (25 ml) and isooctane (1 ml) at −50° C. and the reaction mixture is maintained at the same temperature for 2 hours. The resulting product is isolated and chromatographed on silica gel to afford the starting quinone (70 mg) and CoQ₁₀ of the formula I₁₀ (189 mg) (86% trans).

The method forms 14% cis isomer and therefore far from stereo selective. The reaction does not go to completion and results in poor yield and not suitable for industry.

Method 4: From polyprenyl alcohol and quinone nucleus with silica-alumina as catalyst reported in U.S. Pat. No. 3,998,858(1976) as shown in scheme 4

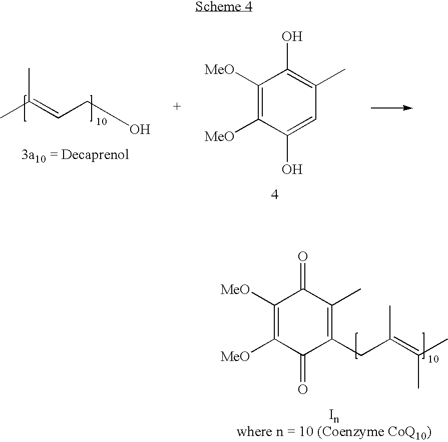

2,3-dimethoxy-5-methyl-1,4-benzohydroquinone of the formula 4, (11 g) is reacted with boric acid (3.6 g) in toluene and water removed azeotropically. The residue is treated with silica-alumina (17 g) and a solution of decaprenol (14 g in 10 ml hexane, 94% purity) and stirred for 1 hour at 30° C. The adsorbent is removed and the filtrate is washed with water, and concentrated, and extracted in ether. The ethereal extract is treated with silver oxide (6 g) and allowed to stand overnight. The reaction mixture is filtered and concentrated to form 16.3 g of crude CoQ₁₀, which is purified by column chromatography, followed by crystallization with acetone to give 8.5 g of CoQ₁₀ of the formula I₁₀ (Lit. mp 49° C.).

The melting point value indicates that process may form a stereoselective process using a simple technique of silica-alumina. However the ratio of silica and alumina to be used and also the respective grades would be critical for the reaction and is not mentioned. The inventors of the present invention tried various grades of silica-alumina and found that the reaction does not proceed.

Method 5: Polyprenyl alcohol and quinone nucleus reported in Chemistry Letters 1597(1988), as shown in scheme 5

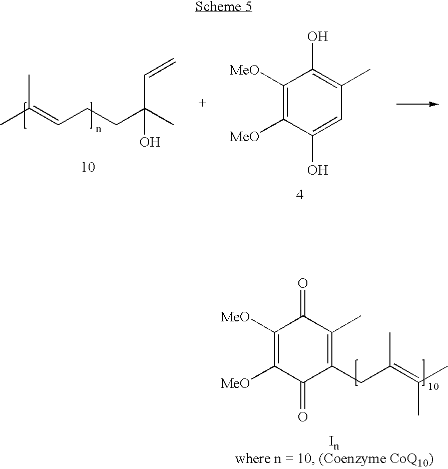

Isodecaprenol compound of the formula 10 (38.8 g, 72% purity) is reacted with 2,3 dimethoxy 5 methyl 1,4 benzohydroquinone compound of formula 4 (75.1 g) in the presence of borontrifluoride etherate in hexane and nitromethane at 43° C. The reaction mixture is quenched in aqueous medium and the nitromethane and the hexane layer is separated. The hexane layer is oxidized with ferric chloride hexahydrate in isopropanol at room temperature. The crude CoQ₁₀ of the formula I₁₀ is obtained in 51% yield with 8% Z isomer

The process forms 8% cis isomer and therefore not stereo selective. Boron trifluoride etherate is a corrosive chemical and not useful for commercialisation.

Thus literature does not provide a stereoselective process for coupling of the benzoquinone with the polyprenyl side chain for the preparation of Coenzymes Q, namely CoQ₉ and CoQ₁₀. As shown in the coupling reactions mentioned above, 8%-15% of cis isomer is formed.

It was observed that purification of such a mixture to get the desired all-trans isomer of CoQ₉ and CoQ₁₀ with less than 1% cis, results in 25-30% purification loss. This would decrease the overall yield of production of these coenzymes mainly CoQ₉ and CoQ₁₀, thereby making the commercial process of making the Coenzyme Q₉ or Coenzyme Q₁₀ cost ineffective.

Scope of clinical application of coenzymes specially CoQ₁₀ is becoming wider with its increasing broadband use Therefore if a cost effective process is developed for the preparation of COQ₁₀ it will greatly help in making this coenzyme easily and at affordable prices.

Preparation of coenzymes CoQₙ where n represents the number of isoprenyl units, namely CoQ₉ or CoQ₁₀, by the coupling of the two key units viz the “benzoquinone nucleus” and the “polyprenyl side chain” should be a straightforward route. However as discussed in prior art, the attempts with such coupling, results in isomerisation of the polyprenyl chain and the geometrical configuration of the chain is not retained. Therefore, the focus should be on the “stereoselective” coupling reaction of the “benzoquinone nucleus” with the corresponding “polyprenyl side chain” to obtain CoQₙ where n represents the number of isoprenyl units. Such a condensation would enhance the cost effectiveness of the preparation of these coenzymes mainly Q₉ or Q₁₀.

The inventors have observed that a simple, straightforward, stereo selective process for the preparation of coenzyme CoQ₉ or CoQ₁₀ of the formulae I₉ and I₁₀ respectively can be developed, by Grignard coupling of the benzoquinone nucleus and the polyprenyl side chain. For such a coupling the “benzoquinone nucleus” has to be converted to the required Grignard reagent with suitable protecting groups. The protecting groups used in literature for making Grignard reagent of the “benzoquinone nucleus” are methoxyethoxymethyl and methyl of the formula IIb & IIc.

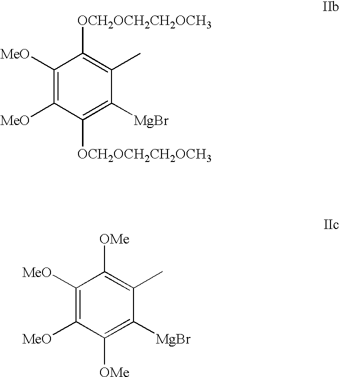

Literature method for making Grignard reagent compound of formula IIb from the compound of the formula 2 as reported in J. Org. Chem. 37 1889 (1972), U.S. Pat. No. 4,270,003 (1981), Synthesis (1981) 469-471 (1982) comprises the methods as depicted in Scheme 6a and Scheme 6b.

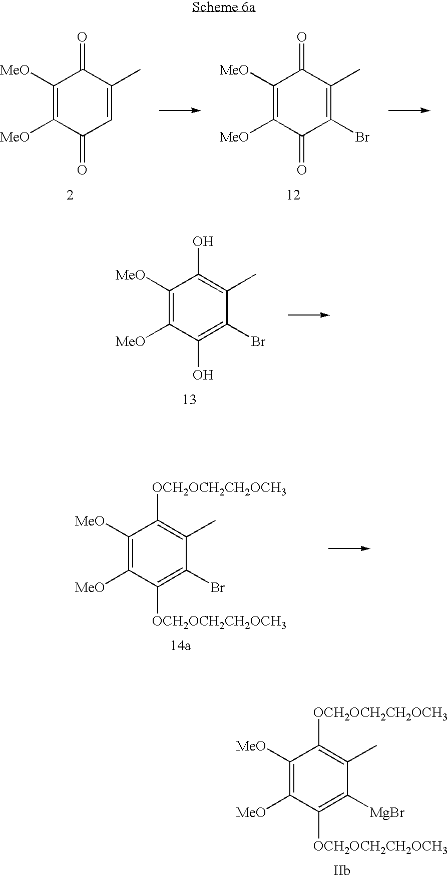

In the method described in the Scheme 6a, 2,3 dimethoxy-5-methyl 1,4 benzoquinone compound of the formula 2 is brominated to form compound of formula 12. The bromination is effected using bromine in carbon tetrachloride and the product of the formula 12 is isolated by washing with ethanol and recrystallizing from petroleum ether, in 74% yield. The compound of the formula 12 is reduced employing aqueous sodium hydrosulphite solution in presence of methanol to get the compound of the formula 13. The compound of the formula 13 is finally converted to compound of the formula 14a by alkylation. The alkylation is carried out in presence of 50% sodium hydride in mineral oil (106 g) which is added in small portions to a stirred solution of 6-bromo-2,3-dimethoxy-5-methyl hydroquinone compound of formula 12 (262.9 g) in 4 litres of N,N dimethyl formamide at −20° C. Chloromethyl 2-methoxyethyl ether (273 g) is added dropwise over a 2 hours period and the mixture is allowed to warm to room temperature. Excess sodium hydride is destroyed with ethanol and the reaction mixture quenched in water. The ethereal layer containing the extracted product is concentrated and the residue purified by column to obtain the compound of formula 14a in 91% yield. The compound of the formula 14a is converted to the compound of the formula IIb, by reacting with magnesium in presence of tetrahydrofuran.

Yield of brominating 3,4 dimethoxy-5-methyl 1,4 benzoquinone, is only 74% which is low for such a simple reaction. The solvent used is toxic and not suitable for scale up. The inventors observed that reduction using aqueous sodium hydrosulphite solution gives yield of the compound of the formula 13 in not more than 40% and therefore not suitable for the industrial production. Further we observed that bromination followed by reduction of the benzoquinone to obtain compound of formula 13, results in low purity of not more than 76%.

The alkylation process uses N,N dimethyl formamide as a solvent and in large excess, 15 times the weight of the bromo compound of the formula 13. N,N dimethyl formamide is a costly solvent and such large excess is not suitable for industry. Sodium hydride used as a base is hazardous and is always present in suspension in oil. The oil also gets extracted in the solvent in which the product compound of formula 14a gets extracted. Thus the process is not compatible to the industry.

Another method of making 2,3 dimethoxy 5-bromo 6-methyl 1,4 hydroquinone is shown in Scheme 6 b

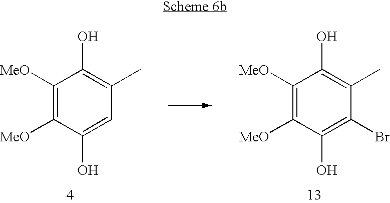

In this method, 2,3-dimethoxy-1,4-hydroquinone of formula 4 is brominated in chloroform at 5° C., and the product isolated from chloroform is in quantitative yield.

We observed that bromination at 5° C. leads to incompletion of reaction and isolation of product from chloroform results in yield less than 75%

The Grignard reagent of formula IIc is prepared as given in scheme 6c

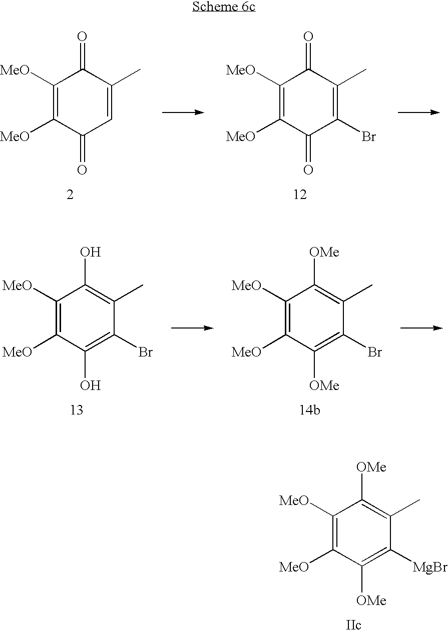

In the process depicted in Scheme 6c, 2,3 dimethoxy 5 methyl benzoquinone of the formula 2 is brominated in room temperature in carbon tetrachloride in 75% yield, reduced with Zinc and acetic acid with 80% yield and methylated with dimethyl sulphate to get the compound of the formula 14b in 62% yield. The compound of the formula 14b is converted to compound of the formula IIc. Yield at each stage of the process is not substantial for mass scale production.

The inventors observed that the above process of reduction with zinc and acetic acid, and methylation after bromination results in purity of compound of formula 14b, which is not more than 76%.

The inventors have found that to avoid the drawbacks of the hitherto known processes exemplified above, the coenzyme CoQ₉ or CoQ₁₀ may be prepared by a simple, straightforward, stereoselective process of coupling of the benzoquinone nucleus with polyprenyl side chain using Grignard reaction of the formula IIb and IIc made by an improved process as more particularly defined hereinafter.

While developing the improved process for the preparation of the Grignard reagents of the formulae IIb and IIc, the inventors developed a new Grignard reagent of the formula IIa.

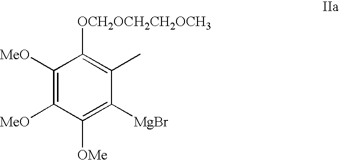

### OBJECTIVE OF THE INVENTION

The main objective of the present invention is to provide an improved process for the stereoselective preparation of the Coenzymes of formula I, namely, CoQ₉ and CoQ₁₀ of the formulae I₉ and I₁₀ respectively as given above.

Another objective of the present invention is to provide an improved process for the preparation of the coenzymes, namely, CoQ₉ and CoQ₁₀ of the formulae I₉ and I₁₀ respectively, which is simple, cost effective and commercially viable.

Still another objective of the present invention is to provide an improved process for the preparation of the coenzymes Q, namely, CoQ₉ and CoQ₁₀ of the formulae I₉ and I₁₀ respectively with high yield (50-56%) and purity 98%

Yet another objective of the present invention is to provide an improved process for the preparation of coenzymes I₉ and I₁₀ by stereospecific coupling of the polyprenyl side chain of formula 3a or 3b_with the Grignard reagents of the formula II.

Still another objective of the present invention is to provide intermediates of the formula III, useful for preparing the coenzymes of formula I.

Still another objective of the present invention is to provide a process for the preparation of intermediates of formula III useful for preparing the coenzyme of formula I.

Still another objective of the present invention is to provide a novel Grignard reagent of the formula IIa useful for preparing the coenzyme of formula I.

Yet another objective of the present invention is to provide a process for the preparation of novel Grignard reagent of the formula IIa useful for the preparation of the coenzymes of formula I.

Yet another objective of the present invention is to provide an improved process for the preparation of Grignard reagents of the formula IIb and IIc useful for the preparation of the coenzymes of formula I.

### SUMMARY OF INVENTION

Thus the present invention relates to an improved process for the preparation of coenzyme of formula I, as shown in scheme A below:

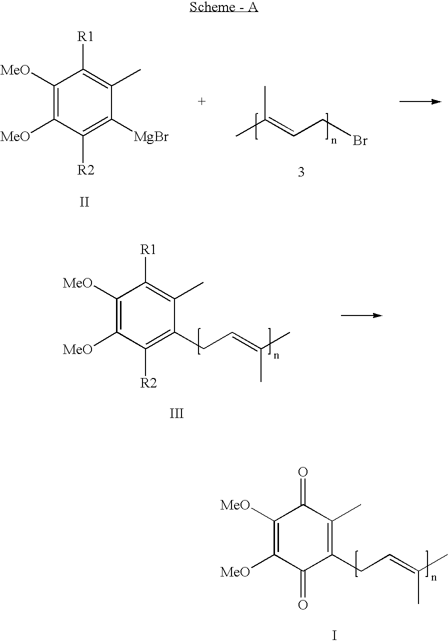

where n is an integer selected from 9 or 10; R1 and R2 are same or different and are selected from —OCH₂_{OCH}₂CH₂OCH₃ or —OMe, with the proviso that when R2 is —OCH₂OCH₂CH₂OCH₃, then R1 is not —OMe.

According to a further aspect of the invention, there is provided preparation of coenzyme CoQ₁₀ (n=10) of the formula I₁₀ as shown in scheme 7 below:

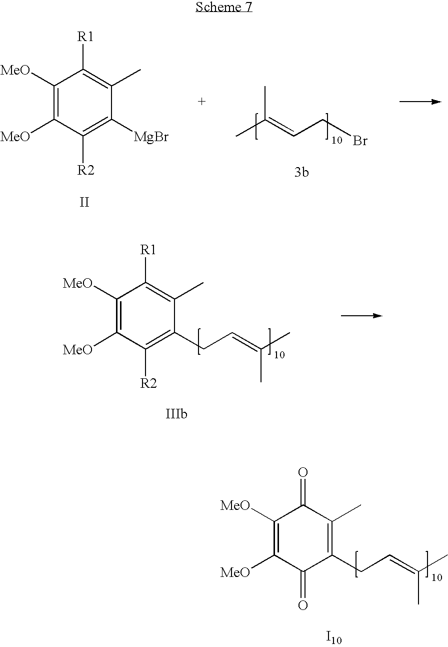

where R1 and R2 are same or different and are selected from —OCH₂OCH₂CH₂OCH₃ or —OMe, with the proviso that when R2 is —OCH₂OCH₂CH₂OCH₃, then R1 is not —OMe

According to still another aspect of the invention, there is provided preparation of coenzyme CoQ₉ (n=9) of the formula I₉ as shown in scheme 8 below:

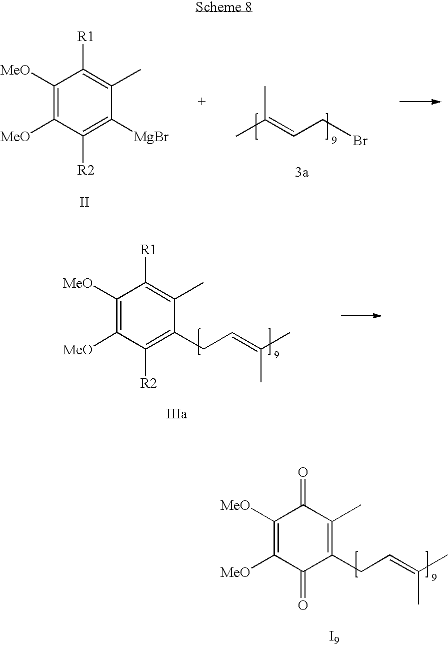

where R1 and R2 are same or different and are selected from —OCH₂OCH₂CH₂OCH₃ or —OMe, with the proviso that when R2 is —OCH₂OCH₂CH₂OCH₃, then R1 is not —OMe

According to yet another aspect of the invention there is provided a novel intermediate of formula III useful for the preparation of coenzymes of formula I

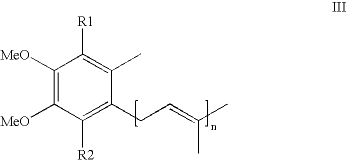

where R1 and R2 are selected from —OCH₂OCH₂CH₂OCH₃ or —OMe, and n is selected from 9 or 10, with the proviso that when R2 is —OCH₂OCH₂CH₂OCH₃, then R1 is not —OMe.

According to yet further aspect of the invention there is provided an improved process for the preparation of compound of formula III, useful for the preparation of coenzymes of formula I

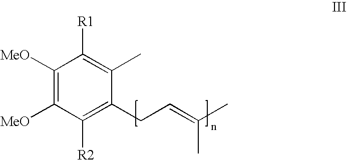

where R1 and R2 are same or different and are selected from —OCH₂OCH₂CH₂OCH₃ or —OMe, and n is selected from 9 or 10, with the proviso that when R2 is —OCH₂OCH₂CH₂OCH₃, then R1 is not —OMe;

which comprises,

i) reacting Grignard reagents of formula II,

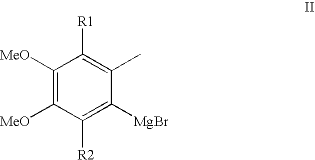

with compounds of formula 3,

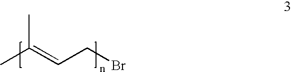

where n is selected from 9 or 10 in presence of cuprous halide in a solvent under inert atmosphere at a temperature in the range of −5° C. to 25° C.

According to another aspect of the invention there is provided a novel Grignard reagent of formula IIa, useful for the preparation of coenzymes of formula I, as shown in scheme 9 below:

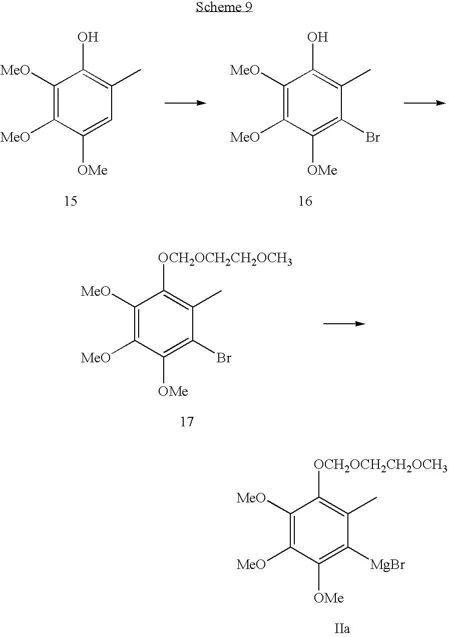

According to a still further aspect of the invention there is provided an improved process for the preparation of Grignard reagent of the formula IIb, useful for the preparation of coenzymes of formula I as shown in scheme 10 below:

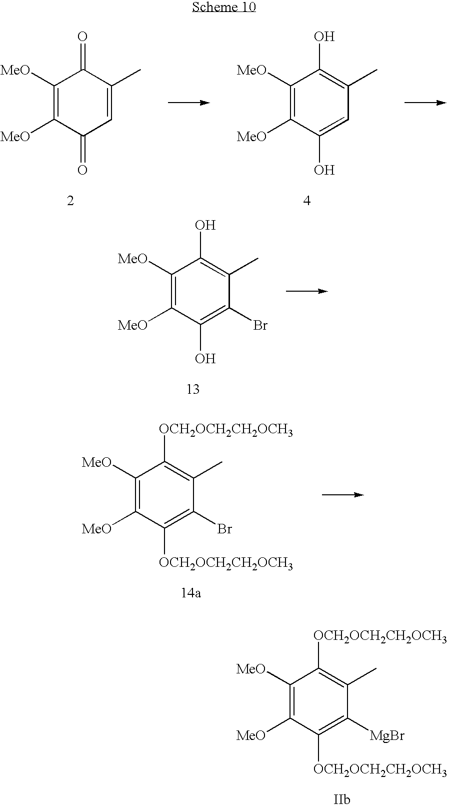

According to a yet further aspect of the invention there is provided a process for the preparation of Grignard reagent of the formula IIc, useful for the preparation of coenzymes of formula I as shown in scheme 11 below:

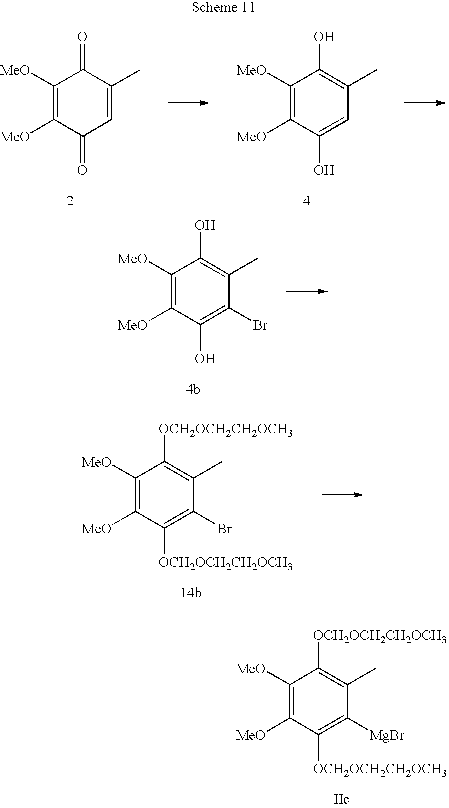

### DETAILED DESCRIPTION

The present invention provides an improved process for the preparation of the coenzymes of formula I, as shown in the Scheme-A

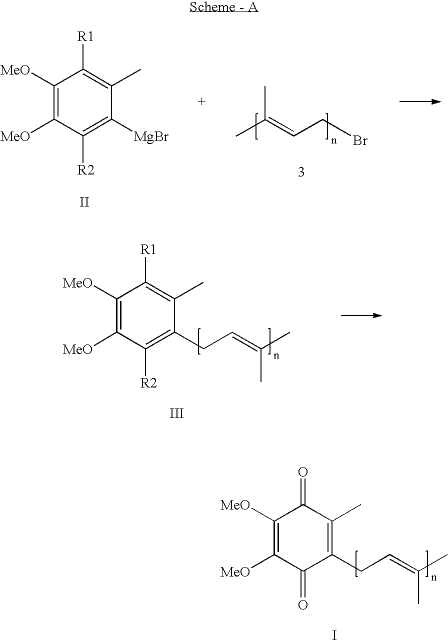

where n is an integer selected from 9 or 10; R1 and R2 are same or different and are selected from —OCH₂OCH₂CH₂OCH₃ or —OMe, with the proviso that when R2 is —OCH₂OCH₂CH₂OCH₃, then R1 is not —OMe.

which comprises,

i) reacting Grignard reagent of formula II,

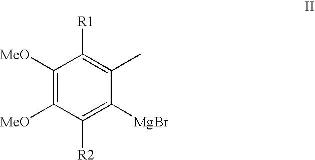

where R1 and R2 are same or different and are selected from —OCH₂OCH₂CH₂OCH₃ or —OMe, with the proviso that when R2 is —OCH₂OCH₂CH₂OCH₃, then R1 is not —OMe;
   with compound of formula 3,

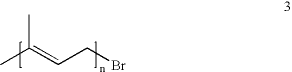

where n is an integer selected from 9 or 10, in presence of cuprous halide in a solvent under inert atmosphere at a temperature in the range of −5° C. to 25° C., to obtain an intermediate of formula III;

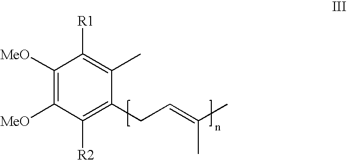

ii) deprotecting the compound of formula III (wherein at least one of R1 and R2 is —OCH₂OCH₂CH₂OCH₃) to obtain the corresponding hydroquinone;

iii) oxidizing the compound of step (i) or (ii) to obtain the coenzyme of formula I;

iv) isolating the compound of formula I; and

v) purifying and crystallizing the coenzyme of formula I by conventional methods.

According to an embodiment of the present invention, there is provided a process for the preparation coenzyme, CoQ₁₀ of the formula I₁₀ as shown in scheme 7:

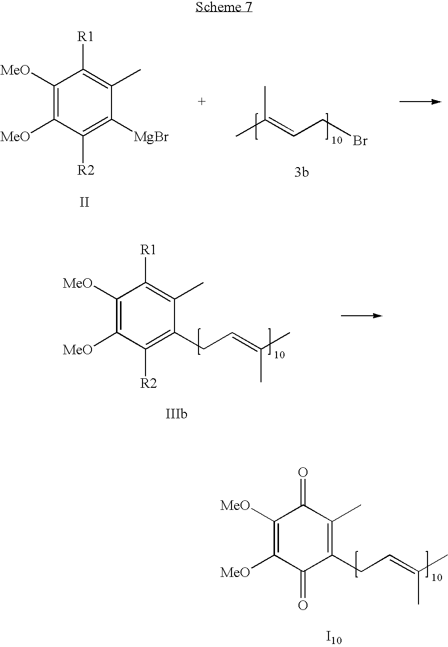

where R1 and R2 are same or different and are selected from —OCH₂OCH₂CH₂OCH₃ or —OMe, with the proviso that when R2 is —OCH₂OCH₂CH₂OCH₃, then R1 is not —OMe

which comprises,

i) reacting Grignard reagent of formula II,

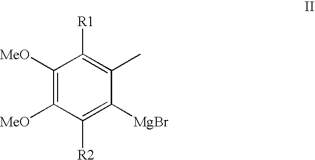

where R1 and R2 are same or different and are selected from —OCH₂OCH₂CH₂OCH₃ or —OMe, with the proviso that when R2 is —OCH₂OCH₂CH₂OCH₃, then R1 is not —OMe;
   with compound of formula 3b,

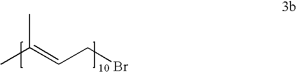

in presence of cuprous halide in a solvent under inert atmosphere at a temperature in the range of −5° C. to 25° C., to obtain an intermediate of formula IIIb;

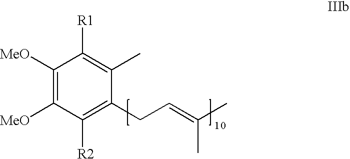

ii) deprotecting the compound of formula IIIb (where at least one of R1 and R2 is —OCH₂OCH₂CH₂OCH₃) to obtain a hydroquinone;

iii) oxidizing the compound of step (i) or (ii) to obtain the coenzyme CoQ₁₀ of formula I₁₀;

iv) isolating the compound of formula I₁₀; and

v) purifying the coenzyme CoQ₁₀ of formula I₁₀ and further crystallizing by conventional method to obtain yellow to orange crystals of the coenzyme CoQ₁₀ of formula I₁₀.

According to another embodiment of the present invention, there is provided a process for the preparation coenzyme, CoQ₉ of the formula I₉ as shown in scheme 8:

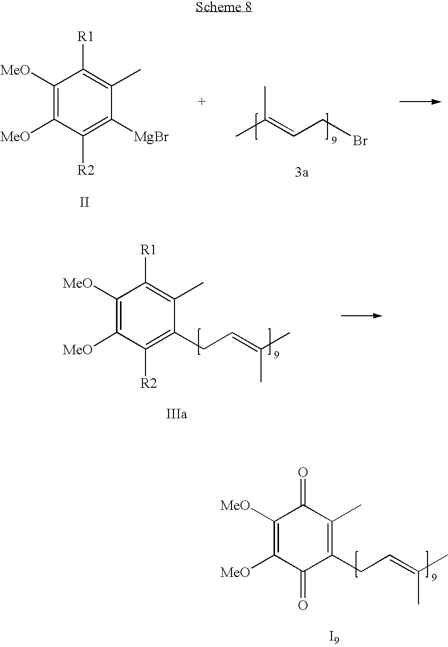

where R1 and R2 are same or different and are selected from —OCH₂OCH₂CH₂OCH₃ or —OMe, with the proviso that when R2 is —OCH₂OCH₂CH₂OCH₃, then R1 is not —OMe

which comprises,

i) reacting Grignard reagents of formula II,

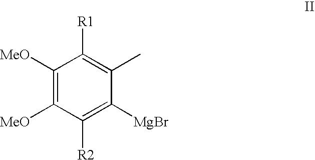

where R1 and R2 are same or different and are selected from —OCH₂OCH₂CH₂OCH₃ or —OMe, with the proviso that when R2 is —OCH₂OCH₂CH₂OCH₃, then R1 is not —OMe;
   with compound of formula 3a,

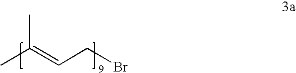

in presence of cuprous halide in a solvent under inert atmosphere at a temperature in the range of −5° C. to 25° C., to obtain an intermediate of formula IIIa;

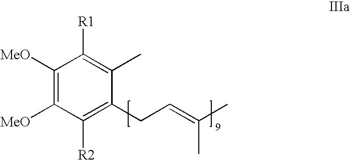

ii) deprotecting the compound of formula IIIa (wherein at least one of R1 and R2 is —OCH₂OCH₂CH₂OCH₃) to obtain a hydroquinone;

iii) oxidizing the compound of step (i) or (ii) to obtain the coenzyme CoQ₉ of formula I₉;

iv) isolating the compound of formula I₉; and

v) purifying the coenzyme CoQ₉ of formula I₉ and further crystallizing by conventional method to obtain yellow to orange crystals of the coenzyme CoQ₉ of formula I₉.

According to still another embodiment of the present invention there is provided novel intermediate of formula III useful in the preparation of coenzymes of formula I

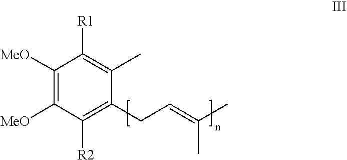

where R1 and R2 are selected from —OCH₂OCH₂CH₂OCH₃ or —OMe, and n is selected from 9 or 10, with the proviso that when R2 is —OCH₂OCH₂CH₂OCH₃, then R1 is not —OMe.

According to yet another embodiment of the present invention, there is provided an improved process for the preparation of intermediates of formula III useful in the preparation of coenzymes of formula I.

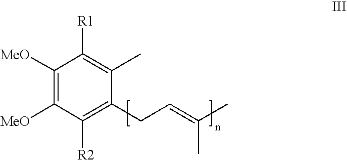

where R1 and R2 are same or different and are selected from —OCH₂OCH₂CH₂OCH₃ or —OMe, and n is selected from 9 or 10, with the proviso that when R2 is —OCH₂OCH₂CH₂OCH₃, then R1 is not —OMe,

which comprises,

i) reacting Grignard reagents of formula II,

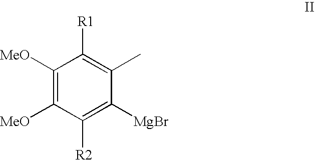

where R1 and R2 are same or different and are selected from —OCH₂OCH₂CH₂OCH₃ or —OMe, with the proviso that when R2 is —OCH₂OCH₂CH₂OCH₃, then R1 is not —OMe;
   with compound of formula 3,

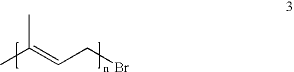

where n is selected from 9 or 10, in presence of cuprous halide in a solvent under inert atmosphere at a temperature in the range of −5° C. to 25° C.

According to still another embodiment of the present invention, there is provided novel Grignard reagent of formula IIa useful in the preparation of coenzymes of formula I

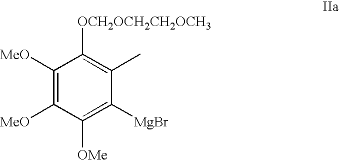

According to yet another embodiment of the present invention, there is provided a process for the preparation of the novel Grignard reagent of the formula IIa, as shown in the Scheme 9

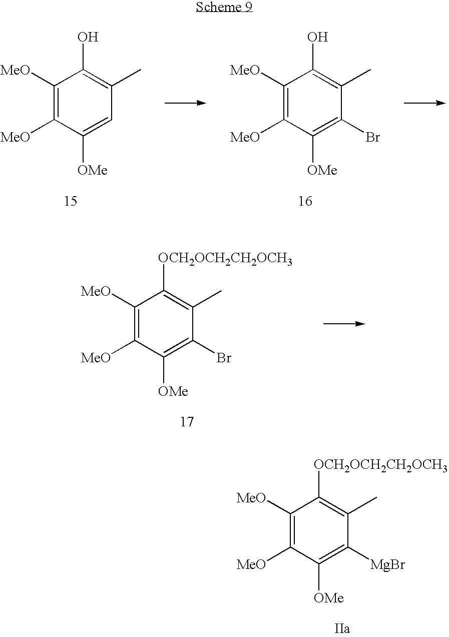

which comprises,

(i) brominating the compound of the formula 15

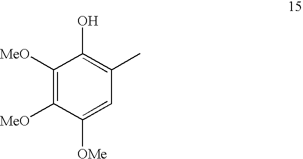

by known method, to obtain compound of formula 16;

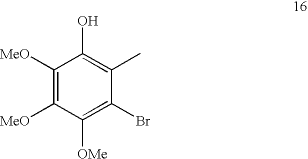

(ii) Alkylating the compound of the formula 16 obtained in step (i) with methoxyethoxymethyl chloride in the presence of a base, an alkali metal alkoxide or metal hydride, to obtain 2,3-dimethoxy-5-methyl-6-bromohydroquinone-1,4-dimethoxyethoxy methyl ether compound of formula 17;

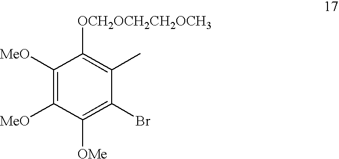

(iii) Reacting the compound of the formula 17 obtained in step (ii) with magnesium in presence of iodine and dibromoethane, using ether as a solvent at a temperature in the range of 0-65° C., to obtain the novel Grignard reagent of the formula IIa;

(iv) cooling the resulting reaction mixture to room temperature, filtering to get the novel Grignard reagent in solution.

The compound of formula 15 can be prepared by methods known in the literature. Synthesis of this novel Grignard reagent is most economical as it can be made from the compound of formula 15, unlike the known Grignard reagents of formula IIb and IIc that are made from 2,3 dimethoxy-5-methyl 1,4 benzoquinone (CoQ₀), thereby having more number of steps in their preparation. Presence of only one protecting group of methoxyethoxymethyl in compound of formula IIa, reduces the requirement of the reagent methoxyethoxyethyl ether as compared to that required in dimethoxyethoxy-methyl ether in IIb, thus making it more cost effective. At the same time cleaving of the protecting group of the formula IIa employed in the present invention results in the formation of the moiety “2,3,4 trimethoxy 6-methyl phenol” that can be easily oxidised with an inexpensive chemical like ferric chloride unlike cerric ammonium nitrate an expensive oxidising agent required for methyl protection when compound of formula IIc is used.

According to still another embodiment of the present invention, there is provided an improved process for the preparation of the Grignard reagent of the formula IIb as shown in Scheme 10

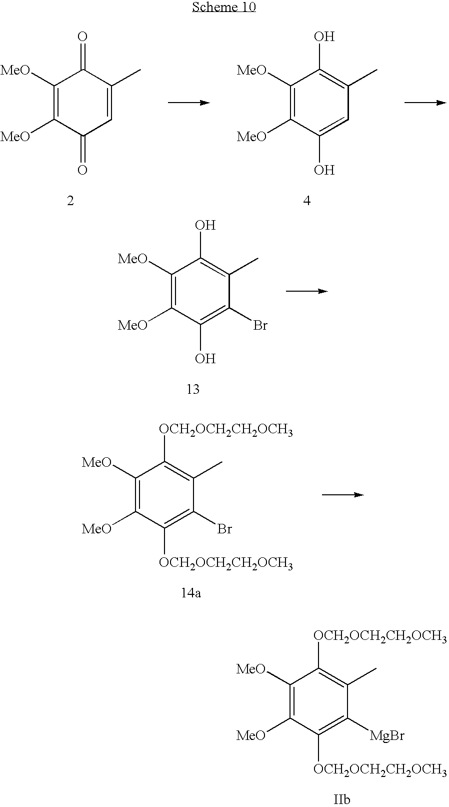

which comprises,

i. Reducing 2,3-dimethoxy-5-methyl-1,4 benzoquinone (CoQ₀) of the formula 2,

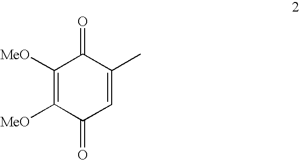

with aqueous sodium hydrosulphite, in alkaline medium, in the presence of a water immiscible organic solvent, separating the organic phase, and evaporating the organic phase to obtain a concentrated residue, to which was added a hydrocarbon solvent to precipitate out compound of formula 4

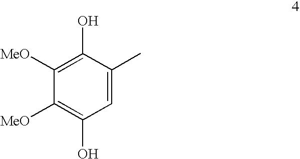

ii. Brominating the resulting compound of the formula 4 with bromine in chlorinated hydrocarbon solvent at a temperature in the range of 0-25° C.,

iii. Quenching the resultant reaction mixture in step (ii) in aqueous medium to obtain aqueous and organic phase, separating the organic phase and evaporating the organic phase to obtain a concentrated residue, to which was added a hydrocarbon solvent to precipitate out 2,3-dimethoxy-5-methyl-6-bromo 1,4 hydroquinone of the formula 13

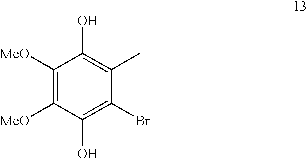

iv. Alkylating the 2,3 dimethoxy-5-methyl-6-bromo 1,4 hydroquinone of the formula 13 obtained in step (iii) with methoxyethoxymethyl chloride in the presence of a base selected from an alkali metal alkoxide or metal hydride, to obtain 2,3-dimethoxy-5-methyl-6-bromo hydroquinone1,4 dimethoxyethoxymethyl ether compound of formula 14a,

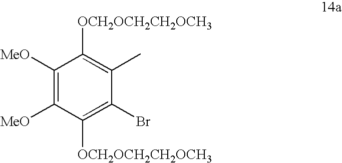

v. Reacting the compound of the formula 14a obtained in step (iv) with magnesium in presence of iodine and dibromoethane, using ether as a solvent at a temperature in the range of 0-65° C., to obtain the Grignard reagent of the formula IIb; and

vi. Isolating the Grignard reagent of formula IIb

Unlike the prior art where reduction in step (i) to obtain compound of formula 4 is effected in homogeneous phase using water miscible solvent, in the process of the present invention, the reduction is carried out using aqueous hydrosulphite, in alkaline medium in the presence of a water immiscible organic solvent, separating the organic phase, and evaporating to obtain a concentrated residue, to which was added a hydrocarbon solvent to precipitate out compound of formula 4 which thereby increases the yield of the reduced product of the formula 4 substantially (to about 96% as compared to about 50% as per the prior art process).

According to the improved process of the present invention, the brominated product of formula 13 was isolated by precipitating out the solid in presence of a hydrocarbon solvent. The process described above increases the yield of the brominated compound (to about 96% as compared to 75% as per the prior art process).

In the modified process of the present invention the alkylation is carried out in the presence of a base sodium hydride in an inexpensive hydrocarbon solvent, or nonhazardous sodium alkoxide, in an inexpensive solvent like alcohol. Thereby making the process economical as compared to prior art where sodium hydride is used in presence of N,N dimethyl formamide which is an expensive solvent.

The bromo compound of formula 14a is reacted with magnesium in the presence of ether selected from diethylether, diisopropyl ether, tetrahydrofuran, at a temperature in the range of 0-65° C., to provide Grignard reagent of the formula IIb having_{—}92% purity.

According to yet another embodiment of the present invention, there is provided an improved process for the preparation of the Grignard reagent of the formula IIc as shown in Scheme 11

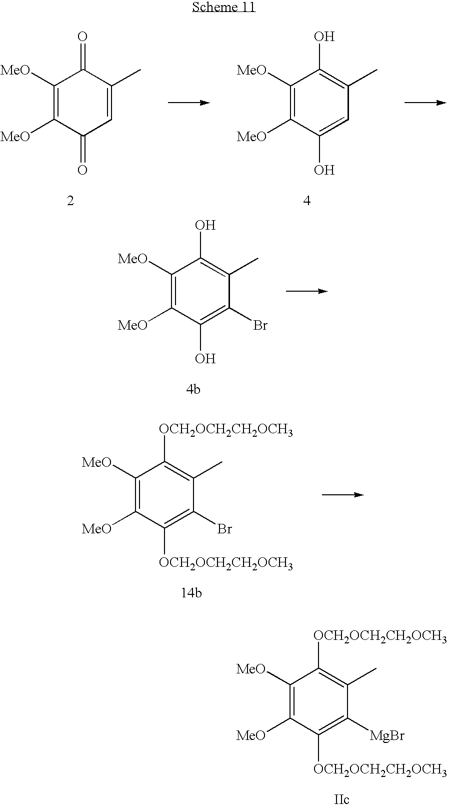

which comprises,

(i) Reducing 2,3 dimethoxy-5-methyl 1,4 benzoquinone (CoQ₀) of the formula 2

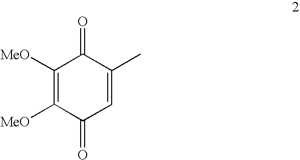

with aqueous sodium hydrosulphite, in alkaline medium, in the presence of a water immiscible organic solvent, separating the organic phase and evaporating the organic phase to obtain a concentrated residue, to which was added a hydrocarbon solvent to precipitate compound of formula 4;

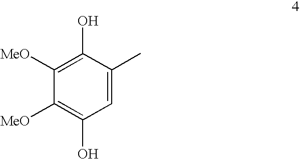

ii. Alkylating the compound of the formula 4, with alkyl sulphate by known method to obtain 2,3,4,5 tetramethoxy toluene compound of formula 4b

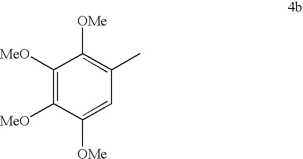

iii. Brominating the resulting compound of the formula 4b with bromine in chlorinated hydrocarbon solvent at a temperature in the range of 0-25° C.,

iv Quenching the resultant reaction mixture in step (iii) in aqueous medium to obtain aqueous and organic phase and separating the organic phase, evaporating the organic phase to obtain a concentrated residue to which was added a hydrocarbon solvent to precipitate out 2,3,4,5 tetramethoxy 6-bromo toluene of the formula 14b

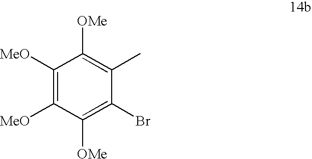

v. Reacting the compound of the formula 14b obtained in step (iv) with magnesium in presence of iodine and dibromoethane, using ether as a solvent at a temperature in the range of 0-65° C., to obtain the Grignard reagent of the formula IIc, and

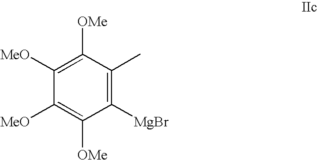

vi. isolating the Grignard reagent of formula IIc.

Unlike the prior art where reduction in step (i) to obtain compound of formula 4 is effected in homogeneous phase using water miscible solvent, in the process of the present invention, the reduction is carried out using aqueous hydrosulphite, in alkaline medium in the presence of a water immiscible organic solvent, separating the organic phase, and evaporating to obtain a concentrated residue, to which was added a hydrocarbon solvent to precipitate out compound of formula 4 which thereby increases the yield of the reduced product of the formula 4 substantially (to about 96% as compared to about 50% as per the prior art process).

According to the improved process of the present invention, the brominated product compound of formula 14b was isolated by precipitating out the solid in presence of a hydrocarbon solvent. The process described above increases the yield of the brominated compound (to about 96% as compared to 75% as per the prior art process).

In the above mentioned process the purity of 2,3,4,5 tetramethoxy 6 methyl bromo benzene of the formula 14b is enhanced when formed by first alkylation of 2,3 dimethoxy 5 methyl 1,4 hydroquinone of the formula 2, to form 2,3,4,5 tetramethoxy toluene compound of formula 4b which can be purified easily by vacuum distillation.

In a preferred embodiment of the present invention the various steps in the processes described above may be carried out as follows,

Reduction of 2,3-dimethoxy 5 methyl 1,4 benzoquinone, CoQ₀ of the formula 2, may be carried out by with sodium hydrosulphite in neutral or alkaline medium, preferably alkaline medium more preferably sodium hydroxide by dissolving CoQ₀ in a water immiscible organic solvent like ether, aromatic hydrocarbons, chlorinated hydrocarbons more preferably chlorinated hydrocarbons like methylene chloride, ethylene chloride, preferably methylene chloride. Thus the reaction may be carried out in biphase, at a temperature in the range of 0° C. to 30° C. preferably, 10 to 20° C. Isolation of 2,3-dimethoxy-5-methyl-1,4-hydroquinone compound of the formula 4, thus formed, may be carried out by acidifying the above reaction mixture, separating the organic phase and concentrating the organic phase. The concentrated organic phase may be added to aliphatic or aromatic hydrocarbon solvent like hexane, heptane, petroleum ether, preferably heptane to precipitate and filter the compound of formula 4.

Bromination of 2,3-dimethoxy-5-methyl-1,4-hydroquinone compound of formula 4, may be carried out with bromine in the presence of a chlorinated hydrocarbon solvent selected from methylene chloride and ethylenechloride at a temperature in the range of 0 to 30° C. preferably 10 to 20° C. Isolation of the brominated compound 2,3-dimethoxy-5-methyl-6-bromo-1,4-hydroquinone of formula 13 thus formed, may be carried out by quenching the resulting reaction mixture in aqueous medium, separating and concentrating the organic phase. The concentrated liquid may be added to a hydrocarbon solvent preferably heptane to precipitate and filter 2,3-dimethoxy-5-methyl-6-bromo-1,4-hydroquinone of formula 13.

Alkylation of 2,3-dimethoxy-5-methyl-6-bromo1,4-hydroquinone of the formula 13 may be carried out with methoxy ethoxy methyl chloride in the presence of metal hydride in aromatic hydrocarbons preferably toluene or an alkali metal alkoxide base selected from sodium methoxide, sodium ethoxide preferably sodium methoxide, in alcohol, at a temperature in the range of −30° C. to 30° C. preferably 15 to 25° C. 2,3-dimethoxy-5-methyl-6-bromo-1,4-hydroquinone methoxyethoxymethyl ether compound of formula 14a thus formed, may be isolated by quenching the reaction mixture in alcohol or aqueous medium, extracting in solvent selected from ether, aromatic hydrocarbon, chlorinated hydrocarbons preferably methylene dichloride, and concentrating the solvent.

2,3-Dimethoxy-5-methyl-6-bromo-1,4-hydroquinone bismethoxyethoxymathyl ether of formula 14a, 2,3,4,5-tetramethoxy-6-methyl-bromo benzene compound of formula 14b or 2,3,4 trimethoxy-5-bromo-6-methyl phenol compound of formula 16 may be converted to the Grignard reagent, as given in literature.

2,3-Dimethoxy-5-methyl-1,4-hydroquinone compound of the formula 4 may be alkylated using dimethylsulphate in acetone or in aqueous medium or in presence of alkali, preferably in aqueous medium in presence of alkali. The resulting product 2,3,4,5 tetramethoxy toluene of formula 4b, may be isolated by extracting in solvent and distilling out the solvent. The resultant residue may be distilled under vacuum at 0.2-10 mm Hg, preferably 0.5-0.8 mm Hg, to obtain the distilled 2,3,4,5 tetramethoxy toluene of formula 4b in more than 96% HPLC purity.

2,3,4,5-tetramethoxy toluene of formula 4b may be brominated as given above to form 2,3,4,5-tetramethoxy-6-methyl bromo benzene of formula 14b.

The coupling of the Grignard reagents of the formula II with solanesyl bromide or decaprenyl bromide of the formula 3a_or 3b may be carried out in the presence of cuprous halide selected from cuprous chloride, cuprous bromide or cuprous iodide preferably cuprous bromide. Grignard reagent may be used in equivalent amount or excess of the solanesyl bromide or decaprenyl bromide in molar ratio of 1:1 to 1:4 preferably 1:1.1 to 1:2. The reaction may be carried out by adding the cuprous salt to the Grignard reagent and allowing to equilibrate for sufficient time. The copper salt is used in 1:1 to 1:0.1 molar ratio of the Grignard reagent. The solanesyl bromide or decaprenyl bromide of the formula 3a or 3b dissolved in a solvent, may be added to the Grignard reagent at temperature range of −25° C. to 25° C. preferably at room temperature. The solvent used may be the same as used for the Grignard reagent or different like aromatic hydrocarbon, aliphatic hydrocarbon like toluene, hexamethylphoshphoric triamide. The solvent for dissolving the solanesyl bromide or decaprenyl bromide may be preferably the same as used in Grignard reaction. The coupling of the Grignard reagent of the formula II, with solanesyl bromide or decaprenyl bromide of the formula 3a_or 3b may also be carried out by adding cuprous salt to the solution of solanesyl bromide or decaprenyl bromide of the formula 3a_or 3b_and the Grignard reagent of the formula II may be added to the above reaction mixture. The reaction may be monitored by HPLC and the rate of addition of the polyprenyl bromide solution may be adjusted with the rate of reaction. The reaction may be quenched in an aqueous medium in acidic or ammonium chloride solution preferably ammonium chloride solution, and the respective product of the formula IIIa or IIIb may be extracted in an water immiscible solvent, solvent evaporated, and the crude compound may be purified by column chromatography to obtain more than 96% pure compound.

Optional deprotection of IIIa (wherein at least one of R1 and R2 is —OCH₂OCH₂CH₂OCH₃) or IIIb (wherein at least one of R1 and R2 is —OCH₂OCH₂CH₂OCH₃) to obtain corresponding hydroquinone may be carried out by method given in literature, followed by oxidation to obtain the final product of compound of formula I₉ or I₁₀.

The oxidation is carried out with cerric ammonium nitrate in acetonitrile as described in literature to obtain the final product of compound of formula I₉ or I₁₀.

The details of the process are given in the Examples below which are provided for illustration only and therefore they should not be construed to limit the scope of the invention

### EXAMPLE 1

### Preparation of Grignard Reagent of 2,3 Dimethoxy-5-bromo-6-methyl 1,4 dimethoxyethoxy methyl ether Compound of Formula IIb

2,3-Dimethoxy 5-methyl-1,4-benzoquinone of formula 2, (2.5 g) was dissolved in 7.5 ml of methylene dichloride and treated with sodium hydrosulphite (3.56 g) in an alkaline solution at 10-20° C. After 2 hours the reaction mixture was treated with conc. HCl (3.4 ml) to acidic pH. The reaction mixture was extracted with methylene dichloride and washed with water. The organic solvent was concentrated and poured in hexane. The precipitated solid was filtered to obtain 2.25 g of 2,3-dimethoxy-5-methyl-1,4-hydroquinone compound of formula 4. The solid was taken in methylene dichloride and treated with bromine (1.96 g) at 10 to 20° C. The reaction was quenched in water after 2 hours and extracted in methylene dichloride. The methylene dichloride was evaporated. The concentrated mass was added to hexane to precipitate out the solid of 2,3-dimethoxy-5-bromo-6-methyl-1,4-hydroquinone (3.06 g). The bromo compound was dissolved in toluene and treated with 1.024 g sodium hydride (60% suspension) in toluene at 0 to −5° C. Methoxyethoxy methyl chloride (3.17 g) was added at 5 to 10° C. The temperature was slowly raised to room temperature and the reaction was continued for 2 hrs. The reaction was quenched with methanol, followed by water and the toluene layer separated. The organic layer was distilled under vacuum to obtain 4.65 g of 2,3-dimethoxy-5-bromo-6-methyl-1,4-hydroquinone dimethoxyethoxy methyl ether compound of the formula 14a. The compound of formula 14a (4.65 g) was reacted with Magnesium (0.301 g) in tetrahydrofuran, in presence of a pinch of iodine at ambient temperature to form the Grignard reagent of 2,3 dimethoxy-5-bromo-6-methyl 1,4 dimethoxyethoxy methyl ether compound of formula IIb

### EXAMPLE 2

### Preparation of Grignard Reagent of 2,3 Dimethoxy-5-bromo-6-methyl 1,4 dimethoxyethoxy methyl ether Compound of Formula IIb

2,3 dimethoxy 5-methyl 1,4 benzoquinone compound of formula 2 (2.5 g) was dissolved in 7.5 ml of methylene dichloride and treated with sodium hydrosulphite (3.56 g) in alkaline solution at 10-20° C. After 2 hours the reaction mixture was treated with conc. HCl 3.4 ml to acidic pH. The reaction mixture was extracted with methylene dichloride and washed with water. The organic solvent was concentrated and poured in hexane (10 ml). The precipitated solid was filtered to obtain 2.25 g of 2,3 dimethoxy 5 methyl 1,4 hydroquinone compound of formula 4. The solid was taken in methylene dichloride 15 ml and treated with bromine (1.96 g) at 10-20° C. The reaction was quenched in water after 2 hours and extracted in methylene dichloride. The methylene dichloride was evaporated. The concentrated mass was added to hexane to precipitate out the solid of 2,3 dimethoxy-5 bromo-6-methyl 1,4 hydroquinone (3.06 g). The bromo compound was dissolved in methanol and treated with sodium methoxide (1.5 g) at 5-10° C. Methoxyethoxy methyl chloride (3.17 g) was added at 5° C.-10° C., the temperature raised to room temperature and maintained for 8 hrs. The reaction was quenched in water and extracted in diisopropyl ether. The organic layer was distilled under vacuum to obtain 4.75 g of 2,3 Dimethoxy-5-bromo-6-methyl 1,4 di methoxyethoxy methyl ether compound of the formula 14a. The compound was reacted with magnesium (0.34 g) in tetrahydrofuran, in presence of a pinch of iodine at ambient temperature to form the Grignard reagent of 2,3 dimethoxy-5-bromo-6-methyl 1,4 dimethoxyethoxy methyl ether of the formula IIb.

### EXAMPLE 3

### Preparation of Grignard Reagent of 2,3,4,5 tetramethoxy-6-methyl-bromobenzene Compound of Formula IIc

2,3dimethoxy-5-methyl 1,4 benzoquinone compound of formula 2, 2.5 g was dissolved in 7.5 ml of methylene dichloride and treated with sodium hydrosulphite (3.56 g) in alkaline solution at 10-20° C. After 2 hours the reaction mixture was treated with conc. HCl (3.4 ml) to acidic pH. The reaction mixture was extracted with methylene dichloride and washed with water. The organic solvent was concentrated and poured in hexane. The precipitated solid was filtered to obtain 2.25 g. of 2,3 dimethoxy 5 methyl 1,4 hydroquinone compound of formula 4. The solid was taken in alkaline solution and dimethyl sulphate (5.75 g) was added at 40-50° C. The reaction mixture was quenched after 4 hours in water and extracted in methylene dichloride. The solvent was evaporated and the crude obtained was distilled under vacuum at 80° C. at 0.5-1.0 mm Hg to obtain 2.33 g of 2,3,4,5-tetramethoxy toluene. The compound was taken in methylene dichloride (15 ml) and treated with bromine (1.75 g) at 10-20° C. The reaction was quenched in water after 2 hours and extracted in methylene dichloride. The methylene dichloride was evaporated. The concentrated mass was added to hexane to precipitate out the solid of 2,3,4,5-tetramethoxy-6-methyl bromobenzene (3.03 g) of formula 14b. The compound of formula 14b was reacted with magnesium (0.30 g) in tetrahydrofuran, at ambient temperature, in presence of a pinch of iodine to form the Grignard reagent 2,3,4,5-tetramethoxy-6-methyl bromobenzene of formula IIc.

### EXAMPLE 4

### Preparation of Grignard Reagent of 2,3,4,5 tetramethoxy-6-methyl-bromobenzene Compound of Formula IIc

2,3-dimethoxy 5-methyl-1,4-benzoquinone of formula 2, (2.5 g) was dissolved in 7.5 ml of methylene dichloride and treated with sodium hydrosulphite (3.56 g) in alkaline solution at 10-20° C. After 2 hours the reaction mixture was treated with conc. HCl (3.4 ml) to acidic pH. The reaction mixture was extracted with methylene dichloride and washed with water. The organic solvent was concentrated and poured in hexane. The precipitated solid was filtered to obtain 2.25 g of 2,3-dimethoxy-5-methyl-1,4-hydroquinone of formula 4. The solid was taken in acetone, potassium carbonate (6.3 g) and dimethyl sulphate (5.75) g were added at 40-50° C. The reaction mixture was quenched after 4 hours in water and extracted in methylene dichloride. The solvent was evaporated and the crude obtained was distilled under vacuum at 80° C. at 0.5-1.0 mm Hg to obtain 2.33 g of 2,3,4,5-tetramethoxy toluene. The compound was taken in methylene dichloride (15 ml) and treated with bromine (1.75 g) at 10-20° C. The reaction was quenched in water after 2 hours and extracted in methylene dichloride. The methylene dichloride was evaporated. The concentrated mass was added to hexane to precipitate out the solid of 2,3,4,5-tetramethoxy-6-methyl-bromobenzene (3.03 g), compound of formula 14b. The compound 14b was reacted with magnesium (0.30 g) in tetrahydrofuran, at ambient temperature, in presence of a pinch of iodine to form the Grignard reagent of 2,3,4,5 tetramethoxy-6-methyl bromobenzene compound of the formula IIc.

### EXAMPLE-5

### Preparation of Novel Grignard Reagent of 2,3,4-trimethoxy-5-bromo-6-methyl-hydroquinone-1-methoxyethoxylmethyl ether of the Formula IIa

2,3,4 trimethoxy-6-methyl-phenol compound of formula 15, (2.42 g) was taken in methylene dichloride 15 ml and treated with bromine 1.96 g at 10-20° C. The reaction was quenched in water after 2 hours and extracted in methylene dichloride. The methylene chloride layer was evaporated. The concentrated mass was added to hexane to precipitate out the solid of 2,3,4 trimethoxy-5 bromo-6-methyl-phenol (3.22 g) of formula 16. The bromo phenol of formula 16 was dissolved in toluene and treated with 0.513 g sodium hydride (60% suspension) in toluene at 0 to −5° C. Methoxyethoxy methyl chloride (1.59 g) was added at 5 to 10° C. The temperature was slowly raised to room temperature and maintained for 2 hrs. The reaction was quenched in water and the toluene layer separated. The organic layer was distilled under vacuum to obtain 4.03 g of 2,3,4-trimethoxy-5-bromo-6-methyl-hydroquinone-1-methoxyethoxylmethyl ether compound of the formula 17. The compound of formula 17 was reacted with magnesium (0.35 g) in tetrahydrofuran, at ambient temperature, in presence of a pinch of iodine, to form the Grignard reagent of 2,3,4-trimethoxy-5-bromo-6-methyl-hydroquinone-1-methoxyethoxylmethyl ether of the formula IIa.

¹H-NMR (300 MHz, CDCl₃, 2.33 (3H, —CH₃), 3.38-3.94 (18H, —OCH₂O—, —CH₂CH₂O—, —OCH₃)

### EXAMPLE 6

### Preparation of Novel Grignard Reagent of 2,3,4-trimethoxy-5-bromo-6-methyl-hydroquinone-1-methoxyethoxylmethyl ether of the Formula IIa.

2,3,4 trimethoxy-6-methyl-phenol compound of formula 15, 2.42 g was taken in methylene dichloride (15 ml) and treated with bromine (1.96 g) at 10 to 20° C. The reaction was quenched in water after 2 hours and extracted in methylene dichloride. The methylene chloride layer was evaporated. The concentrated mass was added to hexane to precipitate out the solid of 2,3,4 trimethoxy-5 bromo-6-methyl-phenol (3.22 g) of formula 16. The bromo phenol of formula 16 was dissolved in methanol and treated with sodium methoxide (0.75 g) at 5-10° C. Methoxyethoxy methyl chloride (1.59 g) was added at 5° C. to 10° C. and the temperature was raised to room temperature and maintained for 8 hrs. The reaction was quenched in water and extracted in diisopropyl ether. The solvent was distilled under vacuum to obtain 4.0 g of 2,3,4-trimethoxy-5-bromo-6-methyl-hydroquinone-1-methoxyethoxylmethyl ether compound of the formula 17. The compound of formula 17 was reacted with magnesium (0.35 g) in tetrahydrofuran, at ambient temperature, in presence of a pinch of iodine, to form the Grignard reagent of 2,3,4-trimethoxy-5-bromo-6-methyl-hydroquinone-1-methoxy-ethoxylmethyl ether of the formula IIa.

¹H-NMR (300 MHz, CDCl₃, 2.33 (3H, —CH₃), 3.38-3.94 (18H, —OCH₂O—, —OCH₂CH₂O—, —OCH₃)

### EXAMPLE 7

### Preparation of Compound of the Formula IIIa (Where R1 and R2=—OCH₂OCH₂CH₂OCH₃)

The Grignard reagent of 2,3 Dimethoxy-5-bromo-6-methyl 1,4 hydroquinone dimethoxyethoxy methyl ether of the formula IIb prepared by the process described in Example 1, was cooled to 0-5° C. Cuprous bromide (0.65 g) was added to the Grignard solution of formula IIb, stirred at room temperature for 1 hour, followed by dropwise addition of a solution of solanesyl bromide in tetrahydrofuran (4 g in 25 ml tetrahydrofuran). The reaction mixture was stirred for four hours and the mixture quenched in 5% ammonium chloride solution and extracted in diethyl ether. The solvent was dried over anhydrous sodium sulphate and evaporated to give 7.2 g of crude, which was purified by column chromatography to give 4.4 g of the pure title compound

### EXAMPLE 8

### Preparation of Compound of the Formula IIIa (Where R1 and R2=—OCH₂OCH₂CH₂OCH₃)

The Grignard reagent of 2,3 Dimethoxy-5-bromo-6-methyl 1,4 dimethoxyethoxy methyl ether compound of the formula IIb prepared by the process described in Example 1, was slowly added to a solution of solanesyl bromide in tetrahydrofuran (4 g in 25 ml tetrahydrofuran) in presence of cuprous bromide (0.65 g). The reaction was continued for four hours at room temperature and the mixture quenched in 5% ammonium chloride solution and extracted in diethyl ether. The solvent was dried over anhydrous sodium sulphate and evaporated to give 7.8 g of crude, which was purified by column chromatography to give 4.0 g of the pure title compound

### EXAMPLE 9

### Preparation of Compound of the Formula IIIa (Where R1 and R2=—OMe)

The Grignard reagent of 2,3,4,5 tetramethoxy-6-methyl bromobenzene compound of the formula IIc, prepared by the process described in Example 3, was cooled at 0-5° C. Cuprous bromide (0.75 g) was added to the Grignard solution of formula IIc, stirred at room temperature for 1 hour, followed by dropwise addition of a solution of solanesyl bromide in tetrahydrofuran (4 g in 25 ml tetrahydrofuran). The reaction mixture was stirred for four hours and the mixture quenched in 5% ammonium chloride solution and extracted in diethyl ether. The solvent was dried over anhydrous sodium sulphate and evaporated to give 7.0 g of crude, which was purified by column chromatography to give 3.78 g of the pure title compound.

### EXAMPLE 10

### Preparation of Compound of the Formula IIIa (Where R1 and R2=—OMe)

The Grignard reagent of 2,3,4,5 tetramethoxy-6-methyl bromobenzene compound of the formula IIc, prepared by the process described in Example 3, was slowly added to a solution of solanesyl bromide in tetrahydrofuran (4 g in 25 ml tetrahydrofuran) in presence of cuprous bromide (0.75 g). The reaction was continued for four hours at room temperature and the mixture quenched in 5% ammonium chloride solution and extracted in diethyl ether. The solvent was dried over anhydrous sodium sulphate and evaporated to give 7.0 g of crude, which was purified by column chromatography to give 3.36 g of the pure title compound.

### EXAMPLE 11

### Preparation of Compound of the Formula IIIa (Where R1=—OCH₂OCH₂CH₂OCH₃ and R2=—OMe)

The Grignard reagent of 2,3,4-trimethoxy-5-bromo-6-methyl-hydroquinone-1-methoxy-ethoxylmethyl ether of the formula IIa prepared by the process described in Example 5, was cooled to 0-5° C. Cuprous bromide (0.79 g) was added to the Grignard solution of formula IIa, stirred at room temperature for 1 hour, followed by dropwise addition of a solution of solanesyl bromide in tetrahydrofuran (4 g in 25 ml tetrahydrofuran). The reaction mixture was stirred for four hours and the mixture quenched in 5% ammonium chloride solution and extracted in diethyl ether. The solvent was dried over anhydrous sodium sulphate and evaporated to give 7.2 g of crude, which was purified by column chromatography to give 4 g of the pure title compound.

### EXAMPLE 12

### Preparation of Compound of the Formula IIIa (Where R1=—OCH₂OCH₂CH₂OCH₃ and R2=—OMe)

The Grignard reagent of 2,3,4-trimethoxy-5-bromo-6-methylhydroquinone-1-methoxy-ethoxylmethyl ether of the formula IIa prepared by the process described in Example 5, was slowly added to a solution of solanesyl bromide in tetrahydrofuran (4 g in 25 ml tetrahydrofuran) in presence of cuprous bromide (0.79 g). The reaction was continued for four hours at room temperature and the mixture quenched in 5% ammonium chloride solution and extracted in diethyl ether. The solvent was dried over anhydrous sodium sulphate and evaporated to give 7.8 g of crude, which was purified by column chromatography to give 3.68 g of the pure title compound.

### EXAMPLE 13

### Preparation of Compound of the Formula IIIb (Where R1 and R2=—OCH₂OCH₂CH₂OCH₃)

The Grignard reagent of 2,3 Dimethoxy-5-bromo-6-methyl 1,4 hydroquinone dimethoxy-ethoxy methyl ether of the formula IIb prepared by the process described in Example 1, was cooled to 0-5° C. Cuprous bromide (0.65 g) was added to the Grignard solution of formula IIb, stirred at room temperature for 1 hour, followed by dropwise addition of a solution of decaprenyl bromide in tetrahydrofuran (4.39 g in 25 ml tetrahydrofuran). The reaction mixture was stirred for four hours and the mixture quenched in 5% ammonium chloride solution and extracted in diethyl ether. The solvent was dried over anhydrous sodium sulphate and evaporated to give 7.2 g of crude, which was purified by column chromatography to give 4.39 g of the pure title compound

### EXAMPLE 14

### Preparation of Compound of the Formula IIIb (Where R1 and R2=—OCH₂OCH₂CH₂OCH₃)

The Grignard reagent of 2,3 Dimethoxy-5bromo-6-methyl 1,4 dimethoxyethoxy methyl ether compound of the formula IIb prepared by the process described in Example 1, was slowly added to a solution of decaprenyl bromide in tetrahydrofuran (4.39 g in 25 ml tetrahydrofuran) in presence of cuprous bromide (0.65 g). The reaction was continued for four hours at room temperature and the mixture quenched in 5% ammonium chloride solution and extracted in diethyl ether. The solvent was dried over anhydrous sodium sulphate and evaporated to give 7.8 g of crude, which was purified by column chromatography to give 3.88 g of the pure title compound.

### EXAMPLE 15

### Preparation of Compound of the Formula IIIb (Where R1 and R2=—OMe)

The Grignard reagent of 2,3,4,5 tetramethoxy-6-methyl bromobenzene compound of the formula IIc, prepared by the process described in Example 3, was cooled to 0-5° C. Cuprous bromide (0.75 g) was added to the Grignard solution of formula IIc, stirred at room temperature for 1 hour, followed by dropwise addition of a solution of decaprenyl bromide in tetrahydrofuran (4.39 g in 25 ml tetrahydrofuran). The reaction mixture was stirred for four hours and the mixture quenched in 5% ammonium chloride solution and extracted in diethyl ether. The solvent was dried over anhydrous sodium sulphate and evaporated to give 7.0 g of crude, which was purified by column chromatography to give 4.11 g of the pure title compound.

### EXAMPLE 16

### Preparation of Compound of the Formula IIIb (Where R1 and R2=—OMe)

The Grignard reagent of 2,3,4,5 tetramethoxy-6-methyl bromobenzene compound of the formula IIc, prepared by the process described in Example 3, was slowly added to a solution of decaprenyl bromide in tetrahydrofuran (4.39 g in 25 ml tetrahydrofuran) in presence of cuprous bromide (0.75 g). The reaction was continued for four hours at room temperature and the mixture quenched in 5% ammonium chloride solution and extracted in diethyl ether. The solvent was dried over anhydrous sodium sulphate and evaporated to give 7.0 g of crude, which was purified by column chromatography to give 3.65 g of the pure title compound.

### EXAMPLE 17

### Preparation of Compound of the Formula IIIb (Where R1=—OCH₂OCH₂CH₂OCH₃ and R2=—OMe)

The Grignard reagent of 2,3,4-trimethoxy-5-bromo-6-methyl-hydroquinone-1-methoxyethoxylmethyl ether of the formula IIa prepared by the process described in Example 5, was cooled to 0-5° C. Cuprous bromide (0.79 g) was added to the Grignard solution of formula IIa, stirred at room temperature for 1 hour, followed by dropwise addition of a solution of decaprenyl bromide in tetrahydrofuran (4.39 g in 25 ml tetrahydrofuran). The reaction mixture was stirred for four hours and the mixture quenched in 5% ammonium chloride solution and extracted in diethyl ether. The solvent was dried over anhydrous sodium sulphate and evaporated to give 7.2 g of crude, which was purified by column chromatography to give 4.45 g of the pure title compound.

### EXAMPLE 18

### Preparation of Compound of the Formula IIIb (Where R1=—OCH₂OCH₂CH₂OCH₃ and R2=—OMe)

The Grignard reagent of 2,3,4-trimethoxy-5-bromo-6-methyl-hydroquinone-1-methoxyethoxylmethyl ether of the formula IIa prepared by the process described in Example 5, was slowly added to a solution of decaprenyl bromide in tetrahydrofuran (4.39 g in 25 ml tetrahydrofuran) in presence of cuprous bromide (0.79 g). The reaction was continued for four hours at room temperature and the mixture quenched in 5% ammonium chloride solution and extracted in diethyl ether. The solvent was dried over anhydrous sodium sulphate and evaporated to give 7.8 g of crude, which was purified by column chromatography to give 3.95 g of the pure title compound.

### EXAMPLE 19

### Preparation of CoQ₉ of Formula I₉

The compound of the formula IIIa (4.4 g) prepared by the process described_in Example 7 was treated with 48% HBr solution (0.22 ml), in presence of isopropanol for 4 hours. The isopropanol was distilled off and the residue was taken in n-hexane. The hexane solution was washed with water dried over anhydrous sodium sulphate and distilled under vacuum to obtain 3.56 g of the residue of CoQ₉ dihydroquinone. The dihydroquinone was oxidized with ferric chloride (2.56 g) in 1 ml water, in presence of isopropanol at room temperature for 3 hours. The reaction was quenched in water and extracted in hexane. The hexane layer was dried over anhydrous sodium sulphate and evaporated to give crude CoQ₉. The crude CoQ₉ was crystallized in ethanol, at 10-15° C., to obtain 2.67 g of pure compound, with overall yield from solanesyl bromide as 58%.

### EXAMPLE 20

### Preparation of CoQ₉ of Formula I₉

The compound of the formula IIIa (3.78 g) prepared by the process described in Example 9 was taken in 48 ml of methylene dichloride and treated with a solution 4 g of cerric ammonium nitrate in 25 ml of acetonitrile and 25 ml of water at 0° C. The reaction mixture was quenched in water and extracted in methylene dichloride solution. The methylene dichloride was concentrated under vacuum to obtain crude CoQ₉. The crude CoQ₉ was purified by column chromatography and crystallized in ethanol, at 10-15° C. to obtain 2.34 g of pure compound, with overall yield from solanesyl bromide as 51%.

### EXAMPLE 21

### Preparation of CoQ₉ of Formula I₉

The compound of the formula IIIa (4.0 g) prepared by the process described in Example 11 was treated with 48% HBr solution (0.22 ml), in presence of isopropanol for 4 hours. The isopropanol was distilled off and the residue was taken in n-hexane. The hexane solution was washed with water dried over anhydrous sodium sulphate and distilled under vacuum to obtain 3.24 g of the residue of CoQ₉ hydroquinone. The hydroquinone was oxidized with ferric chloride (2.56 g) in 1 ml water, in presence of isopropanol at room temperature for 3 hours. The reaction was quenched in water and extracted in hexane. The hexane layer was dried over anhydrous sodium sulphate and evaporated to give crude CoQ₉. The crude CoQ₉ was crystallized in ethanol, at 10-15° C., to obtain 2.30 g of pure compound, with overall yield from solanesyl bromide as 50%.

### EXAMPLE 22

### Preparation of CoQ₁₀ of Formula I₁₀

The compound of the formula IIIb (4.39 g) prepared by the process described in Example 13 was treated with 48% HBr solution (0.22 ml), in presence of isopropanol for 4 hours. The isopropanol was distilled off and the residue was taken in n-hexane. The hexane solution was washed with water dried over anhydrous sodium sulphate and distilled under vacuum to obtain 3.56 g of the residue of CoQ₁₀ dihydroquinone. The dihydroquinone was oxidized with ferric chloride (2.56 g) in 1 ml water, in presence of isopropanol at room temperature for 3 hours. The reaction was quenched in water and extracted in hexane. The hexane layer was dried over anhydrous sodium sulphate and evaporated to give crude CoQ₁₀. The crude CoQ₁₀ was crystallized in ethanol, at 10-15° C., to obtain 2.53 g of pure compound, with overall yield from decaprenyl bromide as 51%.

### EXAMPLE 23

### Preparation of CoQ₁₀ of Formula I₁₀

The compound of the formula IIIb_(4.11 g) prepared by the process described in Example 15 was taken in 48 ml of methylene dichloride and treated with a solution 4 g of cerric ammonium nitrate in 25 ml of acetonitrile and 25 ml of water at 0° C. The reaction mixture was quenched in water and extracted in methylene dichloride solution. The methylene dichloride was concentrated under vacuum to obtain crude CoQ₁₀. The crude CoQ₁₀ was purified by column chromatography and crystallized in ethanol, at 10-15° C., to obtain 2.54 g of pure compound, with overall yield from decaprenyl bromide as 51.0%.

### EXAMPLE 24

### Preparation of CoQ₁₀ of Formula

The compound of the formula IIIb (4.45 g) prepared by the process described in Example 17 was treated with 48% HBr solution (0.22 ml), in presence of isopropanol for 4 hours. The isopropanol was distilled off and the residue was taken in n-hexane. The hexane solution was washed with water dried over anhydrous sodium sulphate and distilled under vacuum to obtain 3.89 g of the residue of CoQ₁₀ hydroquinone. The hydroquinone residue was oxidized with ferric chloride (2.56 g) in 1 ml water, in presence of isopropanol at room temperature for 3 hours. The reaction was quenched in water and extracted in hexane. The hexane layer was dried over anhydrous sodium sulphate and evaporated to give crude CoQ₁₀. The crude CoQ₁₀ was crystallized in ethanol, at 10-15° C., to obtain 2.77 g of pure compound, with overall yield from decaprenyl bromide as 55.8%.

### ADVANTAGES OF THE INVENTION

1. Provides Straight forward coupling of the “benzoquinone nucleus” with the “polyprenyl side chain” for the preparation of the coenzymes Q namely, CoQ₉ and CoQ₁₀.

2 Provides stereoselective coupling reaction for preparation of coenzymes Q namely, CoQ₉ and CoQ₁₀ by simple Grignard reaction, maintaining the geometrical isomer of the double bond. Controlling cis isomer in the reaction decreases purification loss incurred in removing unwanted cis isomer, thereby making the process cost effective.

3. Provides a novel Grignard reagent compound of formula IIa and its preparation, which is useful for the preparation of Coenzymes namely, CoQ₉ and CoQ₁₀.

4. Provides novel intermediates compounds of formula III useful for the preparation of CoQ₉.

5. Provides novel intermediate compounds of formula III useful for the preparation of CoQ₁₀.

1. Process for the preparation of coenzyme of formula I, 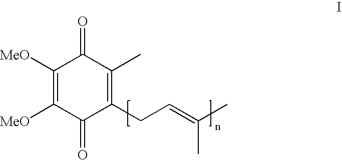
where n is an integer selected from 9 or 10, which comprises,
i) reacting Grignard reagent of formula II, 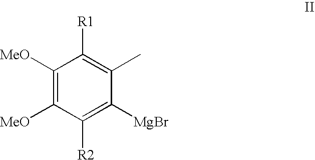
where R1 and R2 are same or different and are selected from —OCH₂OCH₂CH₂OCH₃ or —OMe, with the proviso that when R2 is —OCH₂OCH₂CH₂OCH₃, then R1 is not —OMe;
with compound of formula 3, 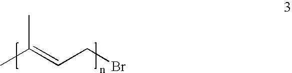
where n is an integer selected from 9 or 10, in presence of cuprous halide in a solvent under inert atmosphere at a temperature in the range of −5° C. to 25° C., to obtain an intermediate of formula III; 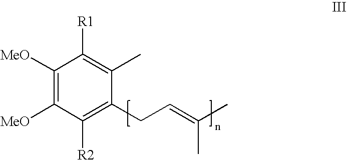
ii) deprotecting the compound of formula III (wherein at least one of R1 and R2 is —OCH₂OCH₂CH₂OCH₃) to obtain the corresponding hydroquinone;
iii) oxidizing the compound of step (i) or (ii) to obtain the coenzyme of formula I;
iv) isolating the compound of formula I; and
v) purifying and crystallizing the coenzyme of formula I by conventional methods.

2. Process as claimed in claim 1, wherein n is 10, for the preparation of coenzyme CoQ₁₀ of the formula I₁₀ 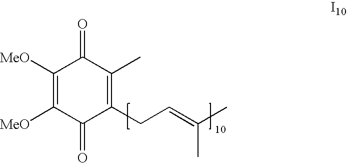
which comprises,
i) reacting Grignard reagents of formula II, 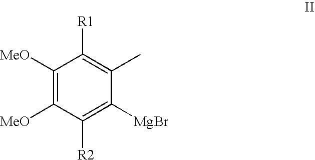
where R1 and R2 are same or different and are selected from —OCH₂OCH₂CH₂OCH₃ or —OMe, with the proviso that when R2 is —OCH₂OCH₂CH₂OCH₃, then R1 is not —OMe;
with compound of formula 3b, in presence of cuprous halide in a solvent under inert atmosphere at a temperature in the range of −5° C. to 25° C., to obtain an intermediate of formula IIIb; 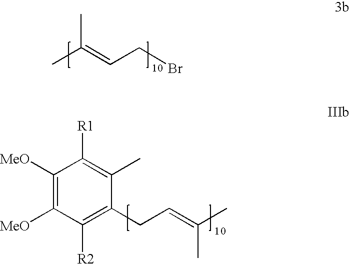
ii) deprotecting the compound of formula IIIb (wherein at least one of R1 and R2 is —OCH₂OCH₂CH₂OCH₃) to obtain the corresponding hydroquinone;
iii) oxidizing the compound of step (i) or (ii) to obtain the coenzyme CoQ₁₀ of formula I₁₀;
iv) isolating the compound of formula I₁₀; and
v) purifying the coenzyme CoQ₁₀ of formula I₁₀ and further crystallizing by conventional method to obtain yellow to orange crystals of the coenzyme CoQ₁₀ of formula I₁₀.

3. Process as claimed in claim 1, wherein n is 9, for the preparation of coenzyme CoQ₉ of the formula I₉ 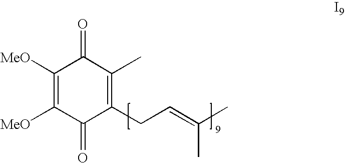
which comprises,
i) reacting Grignard reagents of formula II, 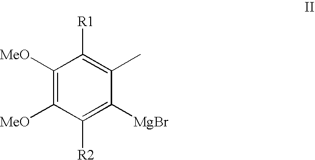
where R1 and R2 are same or different and are selected from —OCH₂OCH₂CH₂OCH₃ or —OMe, with the proviso that when R2 is —OCH₂OCH₂CH₂OCH₃, then R1 is not —OMe;
with compound of formula 3a, in presence of cuprous halide in a solvent under inert atmosphere at a temperature in the range of −5° C. to 25° C., to obtain an intermediate of formula IIIa; 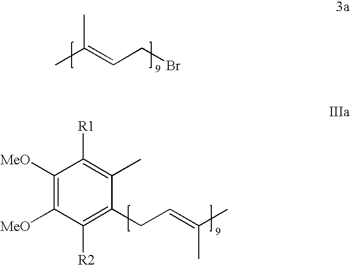
ii) deprotecting the compound of formula IIIa (wherein at least one of R1 and R2 is —OCH₂OCH₂CH₂OCH₃) to obtain the corresponding hydroquinone;
iii) oxidizing the compound of step (i) or (ii) to obtain the coenzyme CoQ₉ of formula I₉;
iv) isolating the compound of formula I₉; and
v) purifying the coenzyme CoQ₉ of formula I₉ and further crystallizing by conventional method to obtain yellow to orange crystals of the coenzyme CoQ₉ of formula I₉.

4. A compound of formula III: 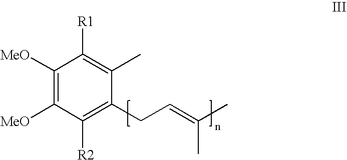
where R1 and R2 are selected from —OCH₂OCH₂CH₂OCH₃ or —OMe, and n is selected from 9 or 10, with the proviso that when R2 is —OCH₂OCH₂CH₂OCH₃, then R1 is not —OMe.

5. Process for the preparation of compound of formula III 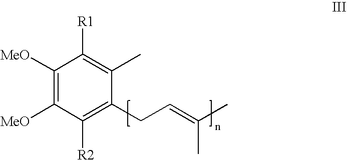
where R1 and R2 are same or different and are selected from —OCH₂OCH₂CH₂OCH₃ or —OMe, and n is selected from 9 or 10, with the proviso that when R2 is —OCH₂OCH₂CH₂OCH₃, then R1 is not —OMe,
which comprises,
i) reacting Grignard reagents of formula II, 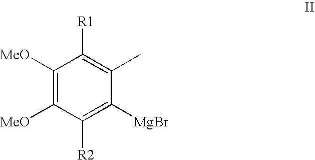
with compounds of formula 3, 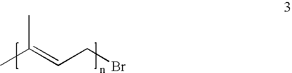
where n is selected from 9 or 10, in presence of cuprous halide in a solvent under inert atmosphere at a temperature in the range of −5° C. to 25° C.

6. Process as claimed in claims 1 and 5 wherein the reaction mixture obtained in step i) is quenched in ammonium chloride solution, and the compound of formula III is extracted in a solvent followed by evaporating the solvent.

7. Process as claimed in claim 6 wherein the extracted compound of formula III is purified by column chromatography to obtain 95% pure compound of formula III

8. Process as claimed in claim 1 and 5 wherein the compound of formula 3 is selected from solanesyl bromide and decaprenyl bromide

9. Process as claimed in claims 1 and 5 wherein the cuprous halide is selected from cuprous chloride, cuprous bromide and cuprous iodide, preferably cuprous bromide in 1:1 to 1:0.1 molar ratio of the Grignard reagent.

10. Process as claimed in claims 1 and 5 wherein the Grignard reagent used is in excess of the compound of formula 3, in a molar ratio of 1:1 to 1:4 preferably 1:1.1 to 1:2.

11. Process as claimed in claim 6 wherein the solvent is selected from water immiscible solvent.

12. Process as claimed in claim 1 wherein step iii) is carried out with cerric ammonium nitrate in acetonitrile.

13. Grignard reagent of formula IIa:

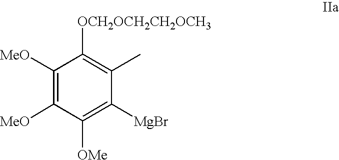

14. Process for the preparation of Grignard reagents of formula IIa as claimed in claim 13, 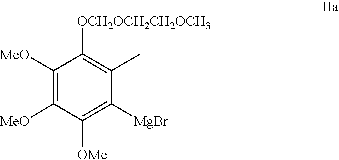
which comprises,
(i) Brominating the compound of the formula 15 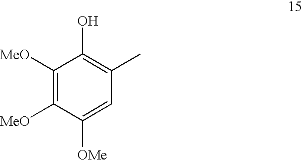
, to obtain compound of formula 16, 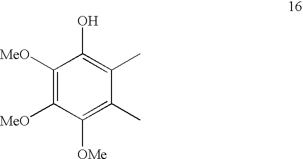
(ii) Alkylating the compound of the formula 16 obtained in step (i) with methoxyethoxymethyl chloride in the presence of a base, an alkali metal alkoxide or metal hydride, to obtain 2,3-dimethoxy-5-methyl-6-bromohydroquinone-1,4 dimethoxyethoxymethyl ether compound of formula 17 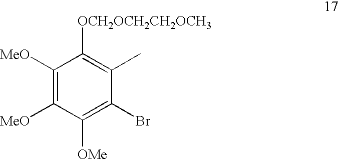
(iii) Reacting the compound of the formula 17 obtained in step (ii) with magnesium in presence of iodine and dibromoethane, using ether as a solvent at a temperature in the range of 0-65° C., to obtain the Grignard reagent of the formula IIa;
(iv) Cooling the resulting reaction mixture to room temperature, filtering to get the novel Grignard reagent of the formula IIa.

15. Process for the preparation of Grignard reagent of the formula IIb, 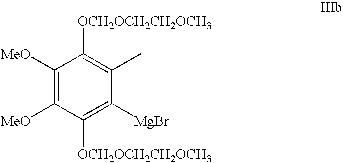
which comprises
Reducing 2,3 dimethoxy-5-methyl 1,4 benzoquinone (CoQ₀) of the formula 2 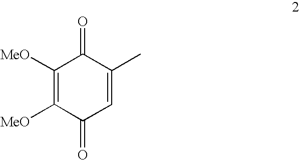
with aqueous sodium hydrosulphite, in alkaline medium, in the presence of a water immiscible organic solvent, separating the organic phase, and evaporating the organic phase to obtain a concentrated residue, to which was added a hydrocarbon solvent to precipitate out compound of formula 4 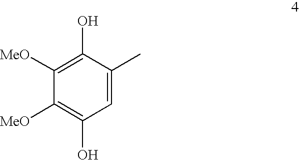
ii. Brominating the resulting compound of the formula 4 with bromine in chlorinated hydrocarbon at 0-25° C.,
iii. Quenching the resultant reaction mixture in step (ii) in aqueous medium to obtain aqueous and organic phase, separating the organic phase and evaporating the organic phase to obtain a concentrated residue, to which was added a hydrocarbon solvent to precipitate out 2,3-dimethoxy-5-methyl-6-bromo 1,4 hydroquinone of the formula 13; 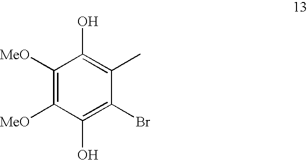
iv. Alkylating the 2,3 dimethoxy-5-methyl-6-bromo 1,4 hydroquinone of the formula 13 obtained in step (iii) with methoxyethoxymethyl chloride in the presence of a base selected from an alkali metal alkoxide or metal hydride, to obtain 2,3-dimethoxy-5-methyl-6-bromo hydroquinone1,4 dimethoxyethoxymethyl ether compound of formula 14a; 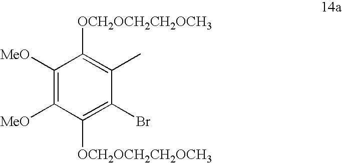
v. Reacting the compound of the formula 14a obtained in step (iv) with magnesium in presence of ether, iodine and dibromoethane, at a temperature in the range of 0-65° C., to obtain the Grignard reagent of the formula IIb; and
vi. Isolating the Grignard reagent of formula IIb

16. Process for the preparation of Grignard reagent of the Formula IIc,_ 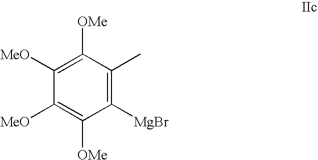
which comprises,
i. Reducing 2,3 dimethoxy-5-methyl 1,4 benzoquinone (CoQ₀) of the formula 2 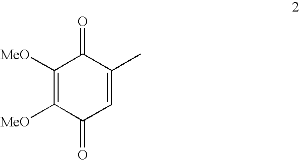
with aqueous sodium hydrosulphite, in alkaline medium, in the presence of a water immiscible organic solvent, separating the organic phase and evaporating the organic phase to obtain a concentrated residue, to which was added a hydrocarbon solvent to precipitate compound of formula 4; 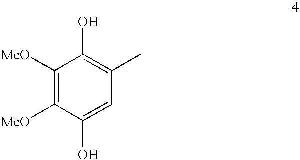
ii. Alkylating the compound of the formula 4, with alkyl sulphate by known method to obtain 2,3,4,5 tetramethoxy toluene compound of formula 4b; 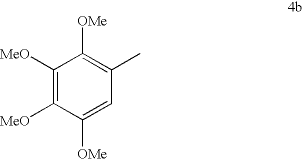
iii. Brominating the resulting compound of the formula 4b with bromine in chlorinated hydrocarbon at a temperature in the range of 0-25° C.;
iv. Quenching the resultant reaction mixture in step (iii) in aqueous medium to obtain aqueous and organic phase and separating the organic phase, evaporating the organic phase to obtain a concentrated residue to which was added a hydrocarbon solvent to precipitate out 2,3,4,5 tetramethoxy 6-bromo toluene of the formula 14b; 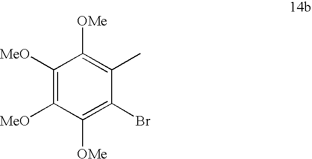
v. Reacting the compound of the formula 14b obtained in step (iv) with magnesium in presence of ether, iodine and dibromoethane, at a temperature in the range of 0-65° C., to obtain the Grignard reagent of the formula IIc; and 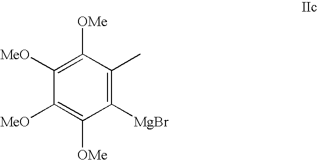
vi. isolating the Grignard reagent of formula IIc.

17. Process as claimed in claim 15 or 16 wherein the reduction of 2,3 Dimethoxy 5 methyl 1,4 benzoquinone, CoQ₀ of the formula 2, is carried out using sodium hydrosulphite in neutral or alkaline medium, preferably alkaline medium more preferably sodium hydroxide at a temperature in the range of 0° C. to 20° C. preferably, 10-20° C.

18. Process as claimed in claim 15 or 16 wherein the water immiscible solvent is selected from water immiscible organic solvent like ether, aromatic hydrocarbons, chlorinated hydrocarbons more preferably chlorinated hydrocarbons like methylene chloride, ethylene chloride, preferably methylene chloride.

19. Process as claimed in claim 15 or 16 wherein the isolation of 2,3 Dimethoxy 5 methyl 1,4 Hydroquinone compound of the formula 4 is effected by acidifying the above reaction mixture of step iv, separating the organic phase, concentrating the organic phase, and adding the concentrated residue to aliphatic or aromatic hydrocarbon solvent like hexane, heptane, petroleum ether, preferably heptane to precipitate and filter the compound of formula 4.

20. Process as claimed in claim 15 or 16 wherein the bromination is carried out using bromine in the presence of a chlorinated hydrocarbon solvent like methylene chloride and ethylenechloride at a temperature in the range of 0-30° C. preferably at 10-20° C.

21. Process as claimed in claim 15 wherein the isolation of the brominated compound 2,3 Dimethoxy-5-methyl-6-bromo1,4 hydroquinone compound of formula 13 formed is carried out by quenching the resulting reaction mixture in aqueous medium, separating and concentrating the organic phase at a temperature in the range of 0 to 20° C. preferably at 0-5° C. and adding the concentrated residue to aliphatic or aromatic hydrocarbon solvent like hexane, heptane, petroleum ether, preferably heptane to precipitate and filter the compound of formula 13

22. Process as claimed in claim 15 wherein the alkylation of 2,3 dimethoxy 5 methyl 6 bromo hydroquinone compound of the formula 13 is carried out using methoxy ethoxymethyl chloride in the presence of metal hydride in aromatic hydrocarbons preferably toluene or an alkali metal alkoxide base selected from sodium methoxide, sodium ethoxide preferably sodium methoxide, in alcohol, at a temperature in the range of −30° C. to 30° C. preferably 15-25° C.

23. Process as claimed in claim 15 wherein the 2,3-dimethoxy-5-methyl-6-bromo 1,4 hydroquinone methoxyethoxymathyl ether compound of formula 14a formed is isolated by quenching the reaction mixture in aqueous medium, extracting in solvent selected from ether, aromatic hydrocarbon, chlorinated hydrocarbons preferably methylene dichloride, and concentrating the solvent.

24. Process as claimed in claim 16 wherein Dimethoxy 5 methyl 1,4 Hydroquinone compound of the formula 4 is alkylated using dimethylsulphate in acetone or in aqueous medium in presence of alkali preferably in aqueous medium in presence of alkali.

25. Process as claimed in claim 16 wherein the resulting 2,3,4,5 tetramethoxy toluene compound of formula 4b is isolated by extracting in solvent and distilling out the solvent, and the resulting residue is distilled under vacuum at 0.2-10 mm Hg, preferably 0.5-0.8 mm Hg,

26. (canceled)

27. (canceled)

28. (canceled)
